(19) United States
(12) Patent Application Publication
(10) Pub. No.: US 2019/0330289 A1
(43) Pub. Date: Oct. 31, 2019

(54) GENERATION OF BRAIN AND SPINAL CORD NEURONS, CARDIAC MYOCYTES, RENAL NEPHRONS AND HEPATOCYTES USING REG PEPTIDES, PEPTIDOMIMETICS, SMALL MOLECULES AND STIMULATORY ANTIBODIES TO REG RECEPTOR

(71) Applicant: Claresa Levetan, Bryn Mawr, PA (US)

(72) Inventor: Claresa Levetan, Bryn Mawr, PA (US)

(21) Appl. No.: 16/511,718

(22) Filed: Jul. 15, 2019

Related U.S. Application Data
(63) Continuation-in-part of application No. 13/662,245, filed on Oct. 26, 2012, now Pat. No. 9,511,110.
Application No. 13/662,245 is a continuation of application No. 15/369,685, filed on Dec. 5, 2016.

(51) Int. Cl. C07K 14/47; C07K 16/28; A61K 35/12; A61K 38/08

(52) U.S. Cl. CPC C07K 14/47; C07K 16/2839; A61K 38/00; A61K 38/08; A61K 35/12

(57) ABSTRACT

Peptides that are bioactive regions and optimized bioactive regions of the human and mammalian Reg gene proteins and that are capable of generation of tissues such as brain, spinal cord, heart, liver, and kidney are described. In particular, 7-15-amino acid Reg peptides and optimized Reg peptides are disclosed which are capable of in vivo and ex vivo transformation of progenitor cells, progenitor tissue and stem cells into specialized cells and tissues, including functioning brain and spinal cord neurons, cardiac myocytes, liver hepatocytes and renal nephrons. Methods of in vivo and ex vivo transformation of progenitor cells into differentiated cells and tissues are also described.

### CROSS-REFERENCE TO RELATED APPLICATIONS

The present application is a Continuation-in-Part (CIP) of U.S. patent application Ser. No. 13/662,245 filed Oct. 26, 2012, which published as U.S. Patent Application Publication No. 2014/0120097 and is now U.S. Pat. No. 9,511,110, the disclosure of which application is incorporated herein by reference in its entirety. The present application is also a Continuation of U.S. patent application Ser. No. 15/369,685 filed Dec. 5, 2016, the disclosure of which application is incorporated herein by reference in its entirety

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created on Nov. 16, 2016 and named C.LEV-017_seqlist1_ST25, which is 16.4 kb in size and which is identical to the paper copy filed with this application.

### FIELD OF THE INVENTION

The present invention relates to generation of brain and spinal cord neurons, including motor neurons, cardiac myocytes, liver hepatocytes, and kidney nephrons by the use of therapies previously patented by this inventor that are bioactive regions and optimized bioactive regions of the human and mammalian Reg gene proteins. These 7-15-amino acid Reg peptides and optimized Reg peptides previously patented by this inventor are specified in this invention for in vivo and ex vivo transformation of progenitor cells, progenitor tissue and stem cells into functioning brain and spinal cord neurons, cardiac myocytes, liver hepatocytes and renal nephrons.

This invention presents data supporting the role of mammalian and human Reg gene peptides ranging in length from 7-15-amino acids and their optimized forms to accelerate the new production of neurons, myocardial cells, nephrons and hepatocytes and for use in conditions in which new neurons and neurite outgrowth may be useful including, but not limited to stroke, acute brain injury, spinal cord injury, Alzheimer's Disease, Fronto-Temporal Atrophy, peripheral neuropathy and other degenerating central and peripheral nervous system diseases; and for the generation of new myocardial cells after myocardial injury and other myocardial injury resulting in a reduction in ejection fraction and may include, but not be limited to congestive heart failure and myocarditis; and new nephrons in conditions including acute and chronic kidney failure and liver disease or injury or post hepatectomy in which new hepatocytes may sustain life or improve quality of life.

In addition to the optimized shorter peptide sequences of the mammalian and human Reg gene proteins that have been previously patented by this inventor, this inventor has identified the human Reg receptor, EXTL-3, and in this patent also identifies a 20-amino acid binding region (SEQ ID NO: 6) within the 919-amino acid Reg receptor/EXTL-3 (SEQ ID NO: 7), from which peptidomimetics, small molecules and stimulatory antibodies are generated to the bioactive region of Reg receptor (SEQ ID NO: 6) that can be derived from either in vivo and ex vivo resulting in production of new brain neurons, spinal cord neurons and motor neurons and which can be used in other neurological conditions to include stroke, spinal cord injury, Alzheimer's Disease, Fronto-Temporal Atrophy, peripheral neuropathy and other degenerating conditions of the central and peripheral nervous system; and for the generation of new myocardial cells after myocardial injury and other myocardial injury including congestive heart failure and myocarditis; new nephrons with acute and chronic kidney failure and new hepatocytes after hepatectomy, liver injury, infection or tumor replacement. Therapies described herein may be used with other medications used in the treatment of neurological, cardiac, renal and hepatic diseases.

It has been known for decades that acute injury to an organ triggers new cell growth by the upregulation of reparative genes as a means of protection. With the advent of the Human Genome, this inventor has identified 100% homologous peptide sequences in the Reg family of gene proteins in man and mammals, which are upregulated in acute injury. Previously, bioactive regions within human and mammalian Reg gene proteins, comprising 7-15-amino acid sequences have been patented by this inventor for their regenerative capacity for use in regeneration of pancreatic islets and beta cells. This inventor has found that during acute injury, not only of the pancreas, but also by the liver, brain, heart and kidney, there is upregulation of the Reg receptor along with upregulation of the Reg gene, which in acute injury present to serve as a Regenerative gene to regenerate healthy cells from progenitor cells present in each organ. Reg gene proteins serve to generate healthy new cells in the pancreas, heart, liver, brain, spinal cord, liver and kidney to protect the organ from cell death.

Previously, this inventor has shown that EXTL-3 is the Reg receptor in man (Levetan C S., et al, Endocr Pract. 2008; 14(9):1075-1083), and that there are 100% homologous—amino acid sequences within the human Reg1a, Reg1b, Reg3a and Reg4 gene proteins. Reg proteins have numerous names in the literature including: Reg1A gene aliases: Regenerating Family Member 1 Alpha, Pancreatic Thread Protein, PTP, Regenerating Islet-Derived Protein 1-Alpha, Islet Of Langerhans Regenerating Protein, Islet Cells Regeneration Factor, Regenerating Protein I Alpha, Pancreatic Stone Protein, PSP, REG-1-Alpha, PSPS1, ICRF, PSPS, REG, Regenerating Islet-Derived 1 Alpha Secretory, Lithostathine 1 Alpha 3, Protein-X 3, P19. Reg proteins from many mammalian species, including man have been shown to interact with the Reg receptor/EXTL-3 and include, but are not limited to the chimpanzee, cow, sheep and mouse. When Reg 7-15-amino acid sequences of the Reg proteins interact with the Reg receptor, EXTL-3, which is a cell surface receptor, the result is downstream activation to the cell nucleus with upregulation of transcription factors resulting in the formation of new functional tissue (Levetan Endocr Pract. 2008; 14(9):1075-1083 and U.S. Pat. Nos. 8,911,776; 8,816,047; 7,989,415; 7,393,919; 9,133,440; 8,829,158; 8,785,400; 8,383,578; 8,211,430; 7,989,412; 7,714,103; 7,393,919; 8,816,047; and 9,321,812).

This inventor and others have shown that Reg gene proteins are typically only expressed during embryonic development when they are critical in the formation of developing organs for the first time during fetal development. After fetal development, the Reg gene proteins are downregulated, but have been found to be highly expressed in times of acute injury (Levetan. J Diabetes. 2010; 2(2):76-84, Levetan Endocr Pract. 2008; 14(9): 1075-83).

New to this invention, is the expression of both Reg genes and their Receptor, EXTL-3, which is typically suppressed after fetal development, but is upregulated in times of acute organ injury, which is not limited to pancreas, but found upregulated and part of the process of repairing the organ including the brain, heart, liver and kidney following acute injury. Previously, Levetan and others found that acute injury to the pancreas resulted in EXTL-3/Reg receptor and Reg expression and use of shorter Reg peptides can interact with the Reg receptor EXTL-3 resulting in new adult cell formation. This invention presents data supporting the role of shorter Reg peptides, rather than using the entire protein sequence, can be used for the transformation of stem cells present in the brain, kidney, heart, and liver following fetal development that can be transformed into new functional neurons, myocardial cells, nephrons and hepatocytes.

This invention identifies a 20-amino acid binding region on the Reg receptor/EXTL-3 (SEQ ID NO: 6), which has 100% homology among man and 17 other mammals, for which SEQ ID NO: 1-5,8-27 interact and small molecules and stimulatory antibodies can be generated for the usage in treatment of neurological, myocardial, renal and hepatic disease or injury, resulting in new neurons, cardiac myocytes, renal nephrons and liver cells and that can be used in a number of medical conditions for which there is currently no treatment to regenerate new tissue within one's own brain, heart, kidney, or liver.

This invention provides new methods for treatment alone or in combination with other agents which may include immune agents and other therapeutics which may improve or maintain symptomatology of mental status, motor capacity, cardiac function including acute myocardial ischemia, myocarditis, diminished ejection fraction, cardiomyopathies and other cardiac conditions, in which improved myocyte mass can improve cardiac function, and quality of life for patients suffering from spinal cord injury, stroke, Alzheimer's Disease, Parkinson's Disease, Fronto-Temporal Atrophy, myocardial infarction, congestive heart failure and chronic or acute liver and kidney disease may be aided.

The findings in this invention of the upregulation of transcription factors including that PDX-1, NGN3, NeurodD1, Pax4, MafA, Nkx2.2, Nkx6.1, B4n4, MafB, Pax6, Nkx6.1 and Sox9 are stimulated by the shorter Reg peptides acting through the Reg receptor, EXTL-3, have been shown by Levetan and others (Levetan. J Diabetes. 2010. 2(2):76-84, Levetan Endocr Pract. 2008; 14(9):1075-83, Kapur Islets. 2012; 4(1):40-8., Li Peptides. 2009; 30(12):2242-9, Assouline-Thomas Differentiation. 2015; 90(4-5):77-90). Mammalian Reg peptides have been shown to induce an up-regulation of proteins involved in nerve regeneration (β-III tubulin, actin and STAT3). Nerve regeneration is marked by characteristic changes in the expression of a number of structural proteins. Reg peptides improve nerve function and enhance regeneration in streptozotocin-induced diabetic C57BL/6 Mice. (Tam J, The FASEB Journal expresses article10.1096/fj.04-1894fje. Published online Sep. 2, 2004.)

Reg proteins have been shown upregulated when there is acute injury to organs including pancreas, liver, brain and myocardium and interact with the Reg Receptor/EXTL-3, and expressed as a protective mechanism during time of injury. This invention specifically identifies Reg peptide sequences previously patented by this inventor, but now for the in vivo development of new cardiac myocytes, brain neurons, nephrons and hepatocytes from the liver, and for ex-vivo expanded neuron, cardiac myocyte, nephrons and hepatocyte to be delivered directly or indirectly to the organ in need.

This inventor demonstrates herein the use of Reg peptides, small molecules, and stimulating antibodies designed to stimulate the 20-amino acid Reg binding site (SEQ ID NO: 6) of the 919-amino acid Reg receptor/EXTL-3 (SEQ ID NO: 9), to be used as therapy in conditions including, but not limited to acute injury to organs, including the brain, central and peripheral nerves, spinal cord injury, stroke, Alzheimer's Disease, Parkinson's Disease, Frontal Temporal Atrophy and other debilitating neurological conditions in which new neurons and neuron outgrowth can be beneficial, acute myocardial ischemia, myocarditis, cardiomyopathy, diminished cardiac ejection fraction, congestive heart failure, injury of the liver from tumors, toxins, cirrhosis, hepatitis or other conditions resulting in hepatic failure or insufficiency, and acute and chronic kidney disease.

This inventor has also raised antibodies to this 20-amino acid binding region of the Reg/EXTL-3 receptor, for which stimulating antibodies and peptidomimetics can be generated to form new cardiac myocytes, new neurons and elongation of existing neurons and hepatocytes resulting from peptides described herein. This invention includes peptidomimetics and stimulatory antibodies to the Reg receptor, EXTL-3 for usage to generate new neurons, nephrons, hepatocytes and cardiac myocytes.

New to the art is that these specific bioactive shorter Reg peptides have been shown by this inventor to interact with the Reg/EXTL-3 receptor and may be used in injured brain, cardiac, kidney and liver tissue, which include motor neurons, spinal cord, peripheral nerves and myocardium. Specifically the peptides may be utilized to regenerate new neurons, neurite outgrowth and serve as a motor neuron mitogen facilitating regeneration after injury, for myocardial cell regeneration in acute stress, to generate new hepatocytes after hepatectomy and liver injury and new nephrons in kidney disease.

Reg gene proteins are typically only expressed during embryonic development when they are critical in the formation of pancreatic islets, neurons, myocardial cells, renal cells and other developing organs being generated for the first time. Expression data demonstrates that after fetal development, the Reg gene proteins and EXTL-3 are downregulated, but have been found to be highly expressed in times of acute injury Levetan Endocr Pract. 2008; 14(9):1075-83, Levetan C., 2010, J Diabetes; 2(2): 76-84) including in the human heart, when the Reg gene protein was shown to be upregulated in response to acute myocardial stress Kiji, Am J Physiol Heart Circ Physiol. 2005; 289(1):H277-84.

Reg gene proteins have been shown to be essential for the survival and neurite outgrowth of spinal cord neurons, with upregulation of the Reg receptor and Reg playing a key a role in protection of spinal cord neurons against injury (Parikh, Biomol Concepts. 2012; 3(1):57-70., Van Ba, J Biol Chem. 2012: 10;287(7):4726-39., Fang. Anat Rec (Hoboken). 2010; 293(3):464-76, Fang, Anat Rec (Hoboken). 2011; 294(1):24-45). Fang has specifically shown the role of Reg in protecting neurons after spinal cord transection to promote functional recovery by increasing axonal growth, inhibiting neuronal apoptosis, and attenuating spinal cord secondary injury after spinal cord injury. (Parikh, Biomol Concepts. 2012; 3(1):57-70., Van Ba, J Biol Chem. 2012: 10;287(7):4726-39., Fang. Anat Rec (Hoboken). 2010; 293(3):464-76, Fang, Anat Rec (Hoboken). 2011; 294(1):24-45).

Reg has been shown to be a motor neuron mitogen facilitating regeneration after injury and promoted neuronal survival, preserved neurite complexity and fasciculation, and protected contents from reactive oxygen species and prevented the formation of cortical and white matter lesions and reduced neuronal death and glial activation following toxic insults (Haldipur. Ann Clin Transl Neurol. 2014; 1(10): 739-54, Simon (FASEB 2003; 17(11): 1441-50 has shown that Reg protein stimulates liver regeneration after partial hepatectomy and combines mitogenic and anti-apoptotic functions. Lieu has shown that Reg both accelerates liver regeneration and protects against acetaminophen injury and Reg inactivation increases sensitivity to hepatotoxicity and delays liver regeneration post-hepatectomy Lieu, Hepatology. 2005; 42(3):618-26. Hepatology. 2006; 44(6):1452-64. Lieu, Hepatology. 2005 September; 42(3):618-26).

Konishi (J Biol Chem. 2013. 12;288(15):10205-13. Konishi, Neuroscience. 2011. 23;175:273-80) also found that Reg protein may serve for regenerating of neurons and neurite elongation and Kawahara found that Reg protein may protect neurons following seizures (Neuroscience. 2011: 23;175:273-80). Namikawa (Biochem Biophys Res Commun. 2005 Jun. 24; 332(1):126-34, Namikawa, The Journal of Neuroscience, Jul. 12, 2006; 26(28):7460-7467), found that the Reg gene is involved in peripheral nerve regeneration and Reg plays pivotal roles during motor neuron and nerve regeneration. Konishi (Neuroscience. 2011. 23;175:273-80) found that Reg 3 gamma formed around neuron injury sites and may service as a scaffold for regenerating axons (Konishi J Biol Chem. 2013. 12;288(15):10205-13).

To date, specifically the shorter peptides and sequences described herein that are bioactive and highly homologous regions of human and mammalian Reg peptides that have been identified and patented by this inventor (U.S. Pat. Nos. 8,911,776; 8,816,047; 9,133,440; 7,989,415; 7,393,919; 9,321,812; 9,133,440; 8,829,158; 8,785,400; 8,383,578; 8,211,430; 7,989,412; 7,714,103; 7,393,919; 8,816,047; and 9,321,812), have not been considered as potential therapy for brain injury, stroke, Alzheimer's Disease, spinal cord injuries, and other central and peripheral nervous system conditions myocardial, renal or liver disease. The bioactive region of the Reg receptor identified in this invention for which antibodies have been raised by this inventor, have not previously been described or considered for the development of stimulatory antibodies or small molecule therapy for binding to the Reg Receptor, EXTL-3, resulting in new neurons, myocytes, hepatocytes and nephrons to be used in treatment of brain injury, stroke, Alzheimer's Disease, Multiple Sclerosis, spinal cord injuries, and other central and peripheral nervous system conditions, and for myocardial, renal and liver diseases.

This invention and sequences described include peptides contained within human Reg1a, Reg1b, Reg3a, Reg 4 and other mammalian peptide sequences for usage in the protection and generation of new neurons, including motor neurons, and myocardial cell protection and regeneration of cardiac myocytes during acute injury, which have not been described previously. Van Ba (J Biol Chem 2012: 10;287(7):4726-39) specifically found that neurite outgrowth with Reg works through the Reg receptor, EXTL-3 and Van Ba noted that when an anti-Reg-antibody to inhibit Reg1a binding to its receptor was used then there diminished neurite outgrowth and concluded that Reg plays a role on neurite outgrowth (Tam, Biochemical and Biophysical Research Communications. 2002; 291: 649-654, The FASEB Journal express article10.1096/fj.04-1894fje. Published online Sep. 2, 2004, Tam, Neuro Report. 2006; 6;17(2):189-93), provides evidence of the effects of INGAP (INGAP peptide), which acts as a mitogen in the peripheral nervous system (PNS), and showed enhanced neurite outgrowth from dorsal root ganglia (DRGs) in vitro suggesting that INGAP peptide promotes Schwann cell proliferation in the DRG which releases trophic factors that promote neurite outgrowth. INGAP shares a close homology with the Reg gene, family of proteins (Rafaeloff, J Clin. Invest. 1997; 99:2100-2109) some of which are expressed in the nervous system, as well as in the similarity in the biology of islet cells and neuronal cells (Livesey, Nature. 1997: 390;614-618, Scharfmann, Horm. Metab Res. 1997: 29; 294-296).

To date, none of the peptides described herein have been described in the prior art for usage in the generation of new neurons, myocytes, nephrons or hepatocytes. While there is demonstration that the entire Reg gene protein improves spinal cord survival (Fang. Anat Rec (Hoboken). 2010; 293(3):464) and serve as a motor neuron mitogen facilitating motor neuron regeneration after injury along with neuron protection, and the whole gene protein was protects and results in new myocyte and hepatocyte generation, the usage of shorter Reg peptides, peptidomimetics to the Reg receptor and small molecules to the Reg receptor are novel to the field of regenerative medicine and have not been considered as potential therapy.

Formulations, derivatives, optimized forms of shorter Reg peptides previously described and patented by this inventor, and the identification of a 20-amino acid binding region (SEQ ID NO:6) on the EXTL-3/Reg receptor by this inventor along with peptidomimetics and stimulating antibodies to the Reg receptor have not previously been used in the treatment of stroke, spinal cord injury, peripheral neuropathy and other progressive diseases of the brain, kidney, heart or liver, all of which express Reg and EXTL-3 receptors in times of acute injury (http://biogps.org/#goto=genereport&id=2137).

These peptides described herein by this inventor have been used to transform progenitor cells within organs that are present after fetal development and available to be transformed into new adult tissues in a given organ that is injured acutely (Levetan Endocr Pract. 2008; 14(9):1075-83). These human peptides including human Reg1a, Reg 3a, and Reg 4a have highly homologous regions to other mammalian Reg gene proteins and have been shown to interact with the Reg receptor/EXTL-3. In acute injury, Reg gene proteins have been found to bind to EXTL-3/Reg receptors in the heart, brain, nephron and liver to generate new heart, brain and liver cells, and whereas this invention identifies the same bioactive binding arm of the mammalian and human Reg peptides that has been shown to interact with the Reg receptor have been shown by this inventor to transforming cells into functioning adult cells through the interaction EXTL-3/Reg receptor. EXTL-3 receptors are found expressed in the brain, heart, pancreas, kidney, liver and other organs (http://biogps.org/#goto=genereport&id=2137), thus targeted therapy in times of acute and chronic injury to these key organs can potentially prolong life and improve quality of life.

The use of Reg peptides, peptidomimetics, small molecules and antibodies targeting the Reg receptor/EXTL-3 20-amino acid binding region (SEQ ID NO: 6), have not previously been described for the usage in ischemic brain injury, neuropathy, Alzheimer's disease, spinal cord injury, peripheral nerve disease, and other central and peripheral nervous system diseases in which new neuron production could improve clinical status, nor in myocardial ischemia and myocardial disease, kidney and liver disease. These same bioactive and homologous regions of the Reg gene proteins in humans and other mammals can also be used for regeneration of neurons, myocytes, nephrons and hepatocytes.

This invention described herein is not contained in the prior art and consist of Reg peptide sequences and optimized peptide sequences including a 15-amino acid optimized mammalian Reg peptide (SEQ ID NO: 8), an optimized 14-amino acid human Reg peptide (SEQ ID NO: 12), a 7-amino acid human Reg peptide, an 8-amino acid human Reg and a 9-amino acid human Reg peptides that have been patented by this inventor (U.S. Pat. Nos. 8,911,776; 8,816,047; 9,133,440; 7,989,415; 7,393,919; 9,321,812; 9,133,440; 8,829,158; 8,785,400; 8,383,578; 8,211,430; 7,989,412; 7,714,103; 7,393,919; 8,816,047; and 9,321,812).

The peptides described in this invention interact with the EXTL-3 cell surface receptor found expressed during the acute injury model in the brain, nephron, heart and liver and result in downstream generation of new cells producing new cardiac myocytes, new neurons, increased neuron length, new nephrons and new liver cells. This invention also describes a specific 20-amino acid binding region (SEQ ID NO: 6) within the Reg Receptor that is contained within its 919-amino acid Reg Receptor (SEQ ID NO: 7), which is the binding site within this invention. This invention also includes small molecules and stimulating antibodies to the 20-amino acid binding site within the 919-amino acid Reg receptor EXTL-3.

This invention also confirms that the Reg protein is found in the acute injury model of the brain, heart, kidney and liver and works through the Reg receptor. The peptides presented in this invention and the specific 20-amino acid binding region within this 919-amino acid human Reg Receptor (SEQ ID NO: 7), have not been described in the prior art for use in acute injury to the brain, spinal column, peripheral or central nervous system, for acute myocardial, kidney or liver injury.

This invention includes the generation of new neurons that are developed from formulations, derivatives, optimized forms including peptidomimetics of the peptides and stimulating antibodies to the Reg Receptor that are designed for the usage in the treatment of stroke, cerebral ischemia, Alzheimer's disease, other conditions of the central nervous system in which there is a deficit of neurons including Fronto-Temporal Atrophy, damage to the spinal cord and other conditions of the peripheral nervous system in which new neurons can improve mentation, or quality of life.

Additionally, this invention includes formulations, derivatives, optimized forms including peptidomimetics of the peptides and stimulating antibodies to the Reg Receptor that can improve cardiac function including acute myocardial ischemia, myocarditis, diminished ejection fraction, cardiomyopathies and other cardiac conditions in which improved myocyte mass can improve cardiac function and quality of life. Additionally, formulations, derivatives, optimized forms including peptidomimetics of the peptides and stimulating antibodies to the Reg Receptor are used for acute and chronic liver injury in which new hepatocytes and in acute and chronic kidney disease for which there are EXTL-3 receptors identified.

Routes of delivery of therapies described include, but are not limited to oral, intravenous, intra-arterial, subcutaneous delivery and intrathecal delivery and through the spinal canal for generation central neurons and for spinal cord injuries. Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions.

### BACKGROUND OF THE INVENTION

With the advent of the Human Genome Project, we have been able to identify the genes expressed during an acute injury to the pancreas. As was shown in by many investigators, ligation of the pancreatic ducts, resulted in the formation of new islets. This inventor demonstrated that the human Reg gene peptides interact with the EXTL-3/Reg receptor. Others in the field have shown that progenitor cells are present that can be transformed into adult cells. For example, within the pancreatic ductal cell population (the extraislet tissue) that comprises 98% of the pancreas mass, there are progenitor cells that can give rise to new functional islets within the human pancreas when there is acute injury (Inada, Proc Natl Acad Sci USA. 2008; 16;105(50):19915-9., Davani, Stem Cells. 2007; 25(12):3215-22). Each organ houses progenitor cells, which can be transformed into functioning adult cells when there is acute injury to an organ.

The ability to upregulate genes such as the Reg genes, when acute injury occurs, results in the ability to protect the injured organ. This protective mechanism within each organ represents an ability of the body to respond to acute injury, but whether or not there is enough regeneration to overcome the injury, depends on the degree of the insult to the organ and the amount of regeneration. For example, the ability to form new neurons may not be enough to overcome destruction of neurons in the case of ischemic injury to the brain. Similarly, the rate of new myocardial cells may not be sufficient to negate the rupture of a plaque within the vasculature of the myocardium to prevent damage beyond repair.

Knowledge of the Reg gene, and now its receptor, harnesses the power of the body's own protective mechanism of organ repair. The ability to accelerate neuron growth in the case of a stroke or spinal cord injury or for the rate of myocardial regeneration to be sufficient to repair myocardial tissue would represent a true breakthrough in the field of regenerative medicine, especially in light of stroke, cardiac disease, are the most serious health issues facing humanity. Cardiac disease is the number cause of death worldwide with 17.3 million deaths each year according to the World Health Organization (https://webcache.googleusercontent.com/search?q=cache:ehXZQtNa6vAJ:https://www.heart.org/idc/groups/ahamahpublic/%40wcm/%40sop/%40smd/documents/downloadable/ucm_470704.pdf+&cd=l&hl=en&ct=clnk&gl=us). In the US, alone, 800,000 people die from cardiovascular disease or stroke. Stroke is the leading cause of serious long-term disability according to the Centers for Disease Control (https://www.cdc.gov/stroke/).

To date, the shorter peptides and sequences, bioactive region of the Reg receptor and peptidomimetics and stimulatory antibodies to the Reg, described herein and patented by this inventor (U.S. Pat. Nos. 8,911,776; 8,816,047; 9,133,440; 7,989,415; 7,393,919; 9,321,812; 8,829,158; 8,785,400; 8,383,578; 8,211,430; 7,989,412; 7,714,103; 7,393,919; 8,816,047; and 9,321,812) have not been considered as potential therapy for brain injury, stroke, Alzheimer's Disease, other brain conditions and spinal cord injuries, myocardial, renal or liver disease, which is the invention herein.

The leading hypothesis of how new adult cells can be formed in both children and adults is based upon the original works of scientists nearly a century ago who identified that in acute organ injury there is a protective effect by the body to regenerate the injured organ. Frederick Banting, discovered insulin in 1921, by clamping the pancreatic ducts to induce the formation of new pancreatic cells. Dr. Banting collected the pancreatic secretions after acute pancreatic ligation and these secretions became known as insulin (Banting F G and Best C H. J Lab Clin Med. 1922; 7:464-472). This work was supported by several earlier scientists, who described that although the population of beta cells is primarily formed during embryogenesis, there is the ability to grow new beta cells postnatally, through a process of transformation of progenitor cells from within adult organs tissue was well established (DeTakats G. Endocrinology. 1930; 14:255-264). Frederick Banting attributes his studies leading to the discovery of insulin on the work of Moses Barron who documented that regeneration of injured pancreatic tissue manifests from the pancreatic ducts (Barron M. Surg Gynec Obstet. 1920; 19:437-448). Prior to the widespread availability of insulin, surgeons performed partial pancreatectomies on diabetic children for beta cell regeneration. (DeTakats G. Endocrinology. 1930; 14:255-264).

The ability to generate fully-functional adult cells, through the differentiation of progenitor cells has now been shown by more than a dozen research groups. Over the past several decades, the regenerating gene (Reg or REG) family has emerged among many species, including humans, as a key initiating factor Levetan C., 2010, J Diabetes; 2(2):76-84. After fetal development when organs are populated with endocrine cells for the first time, the Reg genes are usually undetectable, but are upregulated in response to acute injury.

It is also known from the Human Genome Project that there are many genes that are responsible for the formation of cells of the different organs when organs are populated for the first time during fetal development (such as the brain, heart, kidney and pancreas) and such genes are expressed almost exclusively during embryological development. The regenerating gene family of proteins (Reg) are expressed almost exclusively during embryological development and then only expressed when there is acute injury as a mechanism to help repair the injured organ.

This invention identifies and confirms peptide sequences that are highly conserved and 100% identical within the human Reg 1a, human Reg1b, human Reg3a and human Reg 4 proteins and identical Reg peptide sequences within many other mammalian species. This inventor has previously described the role of 7-15-amino acid Human Reg peptides in pancreatic islet development. This present invention, identifies peptide 7-15-amino acid sequences and optimized sequences of these peptides contained within the human Reg1a, Reg1b, Reg3a and Reg4 and more than a dozen other mammals that interact with the Reg Receptor for the formation of new neurons, cardiac myocytes, nephrons and liver cells.

This invention demonstrates the utility of these peptides, derivatives, peptidomimetics and stimulatory antibodies that have been generated from the specific binding regions of the Reg Receptor, EXTL-3. Others have provided evidence that Reg peptides play a direct role in stimulating new tissue. (Kapur R et al Islets. 2012; 4(1), Watanabe T et al., Proc Natl Acad Sci USA. 1994, 26;91(9):3589-92. Zenilman M E, et al., Pancreas. 1998; 17:256-261.)

Previously, this inventor demonstrated that a human Reg3a gene protein has successfully been administered to human pancreatic ductal tissue devoid of islets resulted in a significant increase in insulin concentrations indicating new beta cell formation resulted a 3-fold rise in total beta cells staining insulin in STZ-rendered diabetic mice. (Levetan C S., et al, Endocr Pract. 2008; 14(9):1075-83.) This inventor has used human pancreatic ductal tissue to transform cells by the use of Reg peptides into new islets via the Reg receptor/EXTL-3, which has been confirmed by others. (Li J, et al. Peptides 2009; 30:2242-9, Assouline-Thomas B G, Diabetes 2008, 57(Supp1;1) A2413. Kapur R, et al, Islets. 2012; 4(1).)

The Section of Islet Cell and Regenerative Biology at Joslin Diabetes Center confirmed that the a 15-amino acid Reg was present in the newest beta cells and islets that were formed directly from branching proliferating extra-islet ducts, which also confirms that mechanism of action of Reg peptide is to form new beta cells from extra-islet exocrine tissue (Guo L et al, Diabetes. 2010, 59(suppl;1) A2589). When Reg is inhibited by the administration of a blocking antibody in an animal model of pancreatic injury, there was attenuated recovery, also confirming that Reg's role is both protective and regenerative during acute pancreatic injury (Viterbo D, et al. JOP. 2009; 10(1):15-23).

The Departments of Beta Cell Regeneration at the Hagedorn Research Institute and Peptide and Protein Chemistry at Novo Nordisk reported a 2-fold increase in the volume of new small islets developing from non-endocrine tissue resulting from the treatment with both the human 14-amino acid human Reg3a peptide, and the 15-amino acid mammalian Reg3gamma peptide. (Kapur R, et al, Islets. 2012; 4(1).) Five days after treatment with both the 14-amino acid human Reg3a peptides, HIP, and the 15-amino acid hamster Reg3gamma peptide, INGAP, there were increased levels of new islet markers necessary for islet formation, including NGN3, NKX6.1, SOX9, and INS, indicating that REG is a catalyst for beta cell neogenesis. (Kapur R, et al, Islets. 2012; 4(1).)

Similar to these findings, other data support that the Reg protein is an initiating factor to downstream regulation of new beta cells (Levetan C., 2010, J Diabetes; 2(2):76-84). For example, when Reg is initially expressed, PDX-1, PAX1, Ngn3, Nkx6.1, Sox9 and Ins, are not expressed, and once Reg is present, PDX-1, PAX1, Ngn3, Nkx6.1, Sox9 and Ins and other beta cell proliferation factors become present demonstrating that Reg activates downstream factors necessary for beta cell regeneration. (Vukkadapu S S Physiol Genomics 2005: 21, 201-211, Kapur R., et al., Islets. 2012; 4(1).)

REG1a, REG1b, REG3a and REG4 belong to the C-lectin family and are randomly clustered on 2p12. Terazono K., et al., J Biol Chem (1988), 263: 2111-2114. They share structural and some functional properties and encode proteins that are members of the Reg family with sequence homology as described in this invention. Their products are secretory proteins of the C-type lectin superfamily that are involved in regeneration and proliferation in acute injury in the brain, heart, kidney, liver and pancreas.

The 3-Dimensional Structures of the human Reg gene proteins are very similar in that each has a similar binding arm that protrudes from the main structure that is the binding region for the Reg Receptor. Human Reg1a contains 166-amino acids. Human Reg1b contains 166-amino acids. Human Reg3a contains 174-amino acids. Human Reg4 contains 158-amino acids and a 175-amino acid mammalian Reg protein.

This invention identifies peptide sequences that are within the human Reg1a, human Reg1b, human Reg3a and human Reg4, and other homologous mammalian peptides that are 7-15-amino acids in length and embodiments thereof, have been shown by this inventor to interact with the Reg receptor. This invention specifically demonstrates that homologous peptides within the human Reg1a, human Reg1b, human Reg3a and Reg4 gene protein and other mammalian Reg peptides interact with the Reg receptor, which results in the acceleration of the generation of adult cells from the progenitor cells within the human brain, kidney heart, pancreas and liver.

Also identified is a bioactive domain within the Reg receptor that is immunogenic and stimulatory antibodies to this binding site have been generated. A putative Reg Receptor initially described in rodents, was found by this inventor to be present in human tissue. The prior art described by this inventor includes 7-15 optimized and native peptides that interact with the Reg receptor and therefore can also be used for acute injury of the heart, brain, kidney and liver with downstream signaling resulting from the Reg peptides interacting with the Reg receptor, as can peptidomimetics and stimulatory antibodies to the bioactive 20-amino acid binding region within the 919-amino acid Reg Receptor/EXTL3. (Levetan C S., et al, Endocr Pract. 2008; 14(9):1075-83.)

This present invention demonstrates that the 7-15-amino acid human and mammalian Reg and optimized Reg peptides identified by this inventor to be used for acute brain injury, spinal cord injury, stroke, conditions in which there is cerebral atrophy such as Fronto Temporal Atrophy, Alzheimer's Disease and other conditions in which new neurons and neuron outgrowth could be helpful and improve functional status and quality of life, along with cardiac conditions including cardiomyopathy and acute myocardial infarction, myocarditis and other conditions in which there is a diminished cardiac ejection fraction that can be improved with new cardiac myocytes.

The Reg Receptor and has been described and known as hereditary Multiple Exostoses Gene Isolog with other names describing this receptor including REG RECEPTOR, Reg Receptor, BOTV, BOTY, DKFZp686C2342, exostoses (multiple)-like 3, Exostosin-like 3, EXT-related protein 1, EXTL1, HHREG RECEPTOR, EXTR1, EXTL3 Glucuronyl-galactosylproteoglycan-4-alpha-N-acetylglucosaminyltransferase, KIAA0519, Multiple exostosis-like protein 3, REGR and RPR, exostoses (multiple-like 3, Glucuronyl-galactosylproteoglycan, 4-alpha-N-acetylglucosaminyltransferase, exostosin-like-3, Hereditary multiple exostoses gene isolog, reg receptor, Multiple exostosis-like protein 3, and EC2.4.1. The receptor was named for its similarities to the Exostoses family of genes by homology screening, but it was specifically noted that this receptor is not derived from the Exostoses (EXT and EXTL) genes. Rather, the Reg Receptor protein was categorized as a member of the Exostosin family because it demonstrates a 52% homology to the 262-amino acid C-terminal of the Exostosin-like 2 protein and a 40% homology with the 242-amino acid C-terminal of the Exostosin-like 1 protein, yet there is no homology of Reg Receptor to the N-terminal regions of Exostosin-1 or 2. (Kobayashi S. et al., Anat. Embryol. 207:11-15, 2003.) The Reg Receptor was initially isolated and described by Van Hul and colleagues in 1998 as a 919-amino acid protein, with a 23-amino acid unique N-terminal region containing a transmembrane domain (residues 28-51) and a short intracellular region at the N terminus. Reg Receptor i15 is located on chromosome 8p21. (Saito T. et al, Biochem Biophys Res Commun. 1988, 242(1):61-66, Van Hul W et al., Genomics. 1998; 47(2):230-7.) The N-terminal region (residues 1-656) of the Reg Receptor has no homology to any other members of this family of genes. The 1.6-kbp cDNA, which was initially isolated in the screening of the rat islet cDNA expression library as a Reg-binding protein, contained the N-terminal region alone (-amino acid residues 1-332) (Kobayashi S. et al., Anat. Embryol. 207:11-15, 2003). Because no other members of the EXT family bind to Reg proteins, the Reg binding domain is shown in this invention within the N-terminal region of Reg Receptor.

This inventor was the first to demonstrate peptides not contained within the human 14-amino acid Reg3a, are contained within the human Reg1a, Reg1b, Reg3a and Reg4 protein and bind to the Reg Receptor. Findings by this inventor demonstrate that the Reg Receptor plays a key regulatory role in cell growth and generation of new cells from cells contained within organs. Further, the present invention identifies the binding region within human Reg1a, human Reg1b, human Reg3a and human Reg4 and a binding domain on Reg Receptor, which are targets for the treatment of diabetes and other diseases for which there is need for new beta cells. The present invention further demonstrates these peptides, which have not been described in the prior art, are unique Reg peptides for binding to the Reg Receptor on the surface and are pivotal for new beta cell formation either via direct usage of the peptide, derivatives, optimized versions, peptidomimetics that bind to Reg Receptor or via stimulating antibodies generated from unique binding sites within the Reg Receptor that generate new cardiac, kidney, nerve and liver cells.

This invention finds the Reg Receptor to be a pivotal receptor and a specific site within the Reg Receptor to be the site of Reg binding resulting in the translocation of the Reg Receptor through the cytoplasm to the nucleus resulting in the generation of new cell growth, acceleration and turnover in the damaged organ. This invention demonstrates the ability to use shorter mammalian and human Reg peptides to interact with the Reg receptor resulting in downstream upregulation of transcription factors resulting in new brain neurons, cardiac myocytes, liver hepatocytes and kidney nephrons, when such organs are injured and there is upregulation of the Reg receptor.

### BRIEF SUMMARY OF THE INVENTION

Embodiments of the present invention provide for novel agents and methods for neuron, cardiac myocyte, nephron and liver cell regeneration agents that have not previously been described using optimized mammalian and human Reg peptides, peptidomimetics to the Reg receptor and stimulatory antibodies to the Reg Receptor and to the 20-amino acid specific binding site (SEQ ID NO: 6) found on the 919-amino acid Reg Receptor (SEQ ID NO: 7) for which antibodies have been raised by this inventor. Agents include homologous human 7-15-amino acid peptide sequences within the Reg1a, Reg1b, Reg3a and human Reg4 and a 15-amino acid optimized mammalian Reg peptides, as well as, stimulatory antibodies generated from a unique 20-amino acid binding region within the 919-amino acid protein human Reg Receptor for the usage of new neurons, cardiac myocytes, nephrons and hepatocytes. This invention includes formulations, derivatives, optimized forms of human peptides described, and also includes peptidomimetics and stimulatory antibodies serving as peptidomimetics that are designed for the usage in the treatment of conditions of acute and chronic organ injury in which Reg gene peptide helps generate new adult cells within the adult organ suffering an insult.

This invention includes methods for cardiac, neuron, nephron or hepatocyte generation and includes both in vivo and ex-vivo beta cell regeneration of cells for treatment of patients with heart disease, myocardial infarction, stroke, spinal cord injury, peripheral neuropathy, acute and chronic kidney disease and liver disease from many causes including toxins, tumors, infections. This invention includes methods for direct delivery of agents specified in this invention for generation of new adult neurons, liver cells within a damaged brain, spinal cord, kidney, heart, liver and provides to patients via direct delivery via arterial delivery via radial or femoral artery to the heart and arterial targeting to liver via injection through the umbilical and portal vessels and intrathecal delivery or within the spinal column for patients with stroke and spinal cord injuries, but may also include targeted oral and intravenous, subcutaneous delivery with organ specific targeting and may include direct administration to the liver, brain, kidney and heart. SEQ ID NOs: 1, 2, 3, 4, 5, 8-27 may be delivered as a peptide or encapsulated as a nanoparticle and delivered via oral delivery, oral targeted delivery to the liver, heart, brain, spinal cord or kidney, or delivered via intravenous, intra-arterial, subcutaneously, intrathecally or directly to the injured organ either as a peptide or nanoparticle.

This inventor identified EXTL-3 as the Reg receptor in man (Levetan Endocr Pract. 2008; 14(9):1075-83). This inventor has also identified a 20-amino acid binding region on the 919-amino acid EXTL-3 Reg receptor from which stimulatory antibodies, monoclonal antibodies, small molecules developed from SEQ ID NO: 6, which result in the formation of new neurons, hepatocytes, myocytes, and renal cells may be may be targeted or delivered directly or indirectly and may be encapsulated as a nanoparticle and delivered via oral delivery, oral targeted delivery to the liver, heart, brain, spinal cord or kidney, or delivered via intravenous, intra-arterial, subcutaneously, intrathecally or delivered directly to the injured organ to the injured organ.

In the first embodiment, the present invention provides for the discovery of specific regenerative bioactive peptide sequences within the human Reg protein that are referred to as embodiments of “Regenerative Peptides,” including the discovery of the following bioactive sequences and their optimized versions which include, but are not limited to the following 7-15-amino acid sequences and optimized sequences within the human and mammalian Reg peptides:

SEQ ID NO: 1

This is the 15-amino acid peptide found within the mammalian Reg proteins.

Ile-Gly-Leu-His-Asp-Pro-Ser-His-Gly-Thr-Leu-Pro-
Asn-Gly-Ser IGLHDPSHGTLPNGS

SEQ ID NO: 2

This is the 14-amino acid peptide found within the human Reg proteins.

Ile-Gly-Leu-His-Asp-Pro-Thr-Gln-Gly-Thr-Glu-Pro-
Asn-Gly IGLHDPTQGTEPNG

SEQ ID NO: 3

This is the 7-amino acid peptide found within the mammalian and human Reg proteins.

WIGLHDP
Trp-Ile-Gly-Leu-His-Asp-Pro

SEQ ID NO: 4

This is the 8-amino acid peptide found within the human and mammalian Reg proteins.

VWIGLHDP
Val-Trp-Ile-Gly-Leu-His-Asp-Pro

SEQ ID NO: 5

This is the 9-amino acid peptide found within the human and mammalian Reg proteins.

NVWIGLHDP
Asn-Val-Trp-Ile-Gly-Leu-His-Asp-Pro

In the second embodiment, this discovery includes a 20-amino acid human specific binding site on the Reg Receptor, EXTL-3, with 100% homology to 17 other mammals (SEQ ID NO: 6) within the 919-amino acid Reg Receptor (SEQ ID NO: 7) from which a stimulatory antibody to the Reg Receptor have been generated from SEQ ID NO: 6)

SEQ ID NO: 6

This is a 20-amino acid peptide found within the human Reg receptor.EXTL-3.

Cys-Lys-Lys-Ser-Ile-Glu-Asn-Ala-Lys-Gln-Asp-Leu-
Leu-Gln-Leu-Lys-Asn-Val-Ile-Ser
CKKSIENAKQDLLQLKNVIS

SEQ ID NO: 7

This is the 919-amino acid human Reg receptor, also known as EXTL-3, exostosin-like 3 (public accession number NP_001431).

MTGYTMLRNGGAGNGGQTCMLRWSNRIRLTWLSFTLFVILVFFPLIAHY
YLTTLDEADEAGKRIFGPRVGNELCEVKHVLDLCRIRESVSEELLQLEA
KRQELNSEIAKLNLKIEACKKSIENAKQDLLQLKNVISQTEHSYKELMA
QNQPKLSLPIRLLPEKDDAGLPPPKATRGCRLHNCFDYSRCPLTSGFPV
YVYDSDQFVFGSYLDPLVKQAFQATARANVYVTENADIACLYVILVGEM
QEPVVLRPAELEKQLYSLPHWRTDGHNHVIINLSRKSDTQNLLYNVSTG
RAMVAQSTFYTVQYRPGFDLVVSPLVHAMSEPNFMEIPPQVPVKRKYLF
TFQGEKIESLRSSLQEARSFEEEMEGDPPADYDDRIIATLKAVQDSKLD
QVLVEFTCKNQPKPSLPTEWALCGEREDRLELLKLSTFALIITPGDPRL
VISSGCATRLFEALEVGAVPVVLGEQVQLPYQDMLQWNEAALVVPKPRV
TEVHFLLRSLSDSDLLAMRRQGRFLWETYFSTADSIFNTVLAMIRTRIQ
IPAAPIREEAAAEIPHRSGKAAGTDPNMADNGDLDLGPVETEPPYASPR
YLRNFTLTVTDFYRSWNCAPGPFHLFPHTPFDPVLPSEAKFLGSGTGFR
PIGGGAGGSGKEFQAALGGNVPREQFTVVMLTYEREEVLMNSLERLNGL
PYLNKVVVVWNSPKLPSEDLLWPDIGVPIMVVRTEKNSLNNRFLPWNE
IETEAILSIDDDAHLRHDEIMFGFRVWREARDRIVGFPGRYHAWDIPHQ
SWLYNSNYSCELSMVLTGAAFFHKYYAYLYSYVMPQAIRDMVDEYINCE
DIAMNFLVSHITRKPPIKVTSRWTFRCPGCPQALSHDDSHFHERHKCIN
FFVKVYGYMPLLYTQFRVDSVLFKTRLPHDKTKCFKFI.

In a third embodiment, this discovery also includes “Optimized Regenerative Peptides” including peptidomimetics which refers to variations of Regenerative Peptides wherein the peptide has been modified to increase the stability, solubility, including formulations that have increased protease resistance, reduced immunogenicity, Tmax and bioavailability compared to the native peptide and/or provide greater ease in administration. In certain aspects, the modifications can include acetylation, amidation, pegylation, and/or cyclization, or any combinations of these.

SEQ ID NO: 8

This is the optimization of the mammalian 15-amino acid Reg peptide by blocking with an n-terminal acetyl group and a c-terminal amide group.

Ac--Ile-Gly-Leu-His-Asp-Pro-Ser-His-Gly-Thr-Leu-
Pro-Asn-Gly-Ser-NH2

SEQ ID NO: 9

This is a pegylated version of the mammalian 15-amino acid Reg peptide

Ac--Ile-Gly-Leu-His-Asp-Pro-Ser-His-Gly-Thr-Leu-
Pro-Asn-Gly-Ser (Peg-40KD-maleimide)-NH2

SEQ ID NO:10

This is a pegylated version of the mammalian 15-amino acid Reg peptide

Peg-40KD-maleimide-3-Mpa-Ile-Gly-Leu-His-Asp-Pro-
Ser-His-Gly-Thr-Leu-Pro-Asn-Gly-Ser-NH2
(3-Mpa = 3-mercaptopropionic acid)

SEQ ID NO:11

This is a cyclized version of the mammalian 15-amino acid Reg peptide to include but is not limited to:

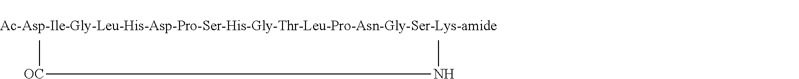

SEQ ID NO: 12

This is the optimization of the 14-amino acid human Reg peptide by blocking with an n-terminal acetyl group and a c-terminal amide group.

Ac--Ile-Gly-Leu-His-Asp-Pro-Thr-Gln-Gly-Thr-Glu-
Pro-Asn-Gly--NH2

SEQ ID NO: 13

This is a pegylated version of the human 14-amino acid Reg peptide

Ac--Ile-Gly-Leu-His-Asp-Pro-Thr-Gln-Gly-Thr-Glu-
Pro-Asn-Gly (Peg-40KD-maleimide)-NH2

SEQ ID NO:14

This is a pegylated version of the mammalian 14-amino acid peptide

Peg-40KD-maleimide-3-Mpa-Ile-Gly-Leu-His-Asp-
Pro-Thr-Gln-Gly-Thr-Glu-Pro-Asn-Gly-NH2
(3-Mpa = 3-mercaptopropionic acid)

SEQ ID NO:15

Cyclized versions of the human 14-amino acid Reg peptide to include but are not limited to:

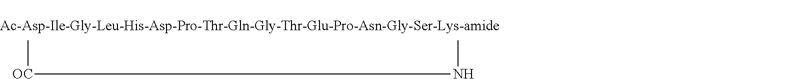

SEQ ID NO: 16

This is the optimization of the 7-amino acid human and mammalian Reg peptide by blocking with an n-terminal acetyl group and a c-terminal amide group.

Ac--Trp-Ile-Gly-Leu-His-Asp-Pro--NH2

SEQ ID NO: 17

This is a pegylated version of the human and mammalian 7-amino acid Reg peptide.

Ac-Trp-Ile-Gly-Leu-His-Asp-Pro (Peg-40KD-
maleimide)-NH2

SEQ ID NO:18

This is a pegylated version of the mammalian and human 7-amino acid Reg peptide.

Peg-40KD-maleimide-3-Mpa-Trp-Ile-Gly-Leu-His-Asp-
Pro-NH2 (3-Mpa = 3-mercaptopropionic acid)

SEQ ID NO:19

Cyclized versions of the human 7-amino acid human and mammalian Reg peptide to include but are not limited to:

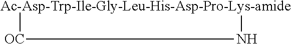

SEQ ID NO: 20

This is the optimization of the 8-amino acid human and mammalian Reg peptide by blocking with an n-terminal acetyl group and a c-terminal amide group.

Ac-Val-Trp-Ile-Gly-Leu-His-Asp-Pro--NH2

SEQ ID NO: 21

This is a pegylated version of the human and mammalian 8-amino acid Reg peptide.

Ac-Val-Trp-Ile-Gly-Leu-His-Asp-Pro (Peg-40KD-
maleimide)-NH2

SEQ ID NO:22

This is a pegylated version of the human and mammalian 8-amino acid Reg peptide

Peg-40KD-maleimide-3-Mpa-Val-Trp-Ile-Gly-Leu-His-
Asp-Pro-NH2 (3-Mpa = 3-mercaptopropionic acid)

SEQ ID NO:23

Cyclized versions of the human and mammalian 8-amino acid Reg peptide to include but are not limited to:

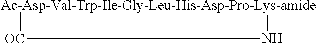

SEQ ID NO: 24

This is the optimization of the 9-amino acid human and mammalian Reg peptide by blocking with an n-terminal acetyl group and a c-terminal amide group.

Ac-Asn-Val-Trp-Ile-Gly-Leu-His-Asp-Pro--NH2

SEQ ID NO: 25

This is a pegylated version of the human and mammalian 9-amino acid Reg peptide

Ac-Asn-Val-Trp-Ile-Gly-Leu-His-Asp-Pro (Peg-40KD-
maleimide)-NH2

SEQ ID NO:26

This is a pegylated version of the human and mammalian 9-amino acid Reg peptide

Peg-40KD-maleimide-3-Mpa-Asn-Val-Trp-Ile-Gly-Leu-
His-Asp-Pro-NH2 (3-Mpa = 3-mercaptopropionic acid)

SEQ ID NO:27

Cyclized versions of the human and mammalian 9-amino acid Reg peptide to include but are not limited to:

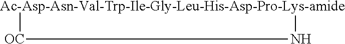

In a fourth embodiment, the in vivo generation of new neurons, cardiac myocytes, nephrons and hepatocytes occurs from the administration of peptidomimetics and includes formulations, derivatives, optimized forms and also include peptides and peptidomimetic agents that bind to the human Reg Receptor and a specific binding region within the Reg Receptor are presented in this invention with the modality of delivery including, but is not limited to: oral, intravenous, subcutaneously, intra-arterial, intrathecal and into the spinal column and targeted or delivered directly or indirectly to the brain, spinal column, heart and liver.

In fifth embodiment, this inventions provides new therapy and includes specific methodology and timing for administration of Regenerative Peptides, Optimized Regenerative Peptides, Regenerative Peptidomimetics and Stimulatory Antibodies to the Reg Receptor and specific methods for generation of new cells for usage, but not limited to generation of neurons for use in stroke, Alzheimer's disease, ischemic brain disease, and other disease of the brain that may be aided by more neurons including fronto-temporal atrophy and other neurological conditions that will be aided and improve quality of life by productions of new neurons; cardiac diseases including acute myocardial infarction, myocarditis, dilated cardiomyopathy and conditions in which additional myocytes will improve cardiac function, reduce congestive heart failure, and improve quality of life for patients with cardiovascular disease; new hepatocytes for usage in patients with liver disease by infection, toxin, hepatic surgery or metastatic or primary cancer of the liver and acute and chronic kidney disease.

In sixth embodiment, an innovative therapy for accelerated generation of the ex-vivo generation of beta cells by methodology for administration of Regenerative Peptides and/or formulations, derivatives, optimized forms including peptidomimetics and stimulatory antibodies to the Reg Receptor that are formed ex-vivo using Regenerative formulations, derivatives, optimized forms including peptidomimetics and stimulating Reg Receptor antibodies for ex-vivo by the transformation of new neurons, new cardiac myocytes and new hepatocytes from adult human tissue of the brain, heart or liver, kidney and also to include pluripotent stem cells including embryonic cells, adult somatic stem cells, human adult bone-marrow derived stem cells, umbilical cord stems cells, mesenchymal stem cells, human amniotic membrane-derived mesenchymal cells, pluripotent cells, mammalian stem cells, mammalian stem cells and ectodermal stem cells that are induced by this invention into new beta cells, neurons, cardiac myocytes, nephrons and hepatocytes that may be administered intravenously, subcutaneously, intra-arterial, intrathecal delivery and delivery into the spinal column and delivery including delivery directly or indirectly to the brain, liver, spinal column and heart that are appropriate targets to optimize efficacy.

In a seventh embodiment, the present invention provide pharmaceutical formulations and unit dose forms of Regenerative Reg Peptides alone or in combination with one or more other active pharmaceutical ingredients (APIs). In one embodiment, the API is an agent in soluble liposome or nanoparticle preparations that allow Regenerative (Reg) Peptides to be administered by a variety of routes, including subcutaneously, intramuscularly, intravenously, intra-arterially, and even orally, depending on the formulation selected. In one embodiment, the formulation comprises a targeting agent for targeted administration to specific locations, receptors, cells, tissues, organs, or organ systems including targeting to the liver, brain, spinal column or heart.

In eighth embodiment, this inventions provides new therapy and specific methods for the transformation of new neurons, new cardiac myocytes and new hepatocytes from adult human tissue of the brain, heart or liver, kidney and also to include pluripotent stem cells including embryonic cells, adult somatic stem cells, human adult bone-marrow derived stem cells, umbilical cord stems cells, mesenchymal stem cells, human amniotic membrane-derived mesenchymal cells, pluripotent cells, mammalian stem cells, mammalian stem cells and ectodermal stem cells that are induced by this invention into new beta cells, neurons, cardiac myocytes, nephrons and hepatocytes that may be administered intravenously, subcutaneously, intra-arterial, intrathecal delivery and delivery into the spinal column and delivery including delivery directly or indirectly to the brain, liver, spinal column and heart that are appropriate targets to optimize efficacy in combination of other therapies, which include medical therapies used for heart, liver, brain and kidney.

### BRIEF DESCRIPTION OF THE DRAWINGS

The file of this patent contains at least one photograph or drawing executed in color. For a fuller understanding of the nature and advantages of the present invention, reference should be made to the following detailed description taken in connection with the accompanying drawings, in which:

FIGS. 1A-C are graphics depicting the 3-dimensional structures of human Reg1a, human Reg3a and Hamster Reg 3gamma by Swiss-Prot folding algorithms with the Regenerative Peptides contained within the Red circled binding arm. Regenerative peptides are contained within the Red circled binding arm.

FIG. 2 is a set of sequences which demonstrate peptide homology between human Reg1a, Reg1b, Reg3a and Reg 4 and the Hamster Reg3gamma. The common sequences are highlighted in red.

FIG. 3 is the 919-amino acid sequence of the Reg Receptor with the 20-amino acid binding domain (SEQ ID NO: 6) presented in this invention is bolded.

FIG. 4 demonstrates the results of studies generating stimulatory antibodies to a 20-amino acid binding site within the Reg Receptor.

FIG. 5 provides the protocol for development of stimulatory antibodies to the binding domains within the Reg Receptor

FIG. 6 is the documentation of titer controls and norms for the antibody studies.

FIGS. 7A-C are images of an SDS PAGE gel and Western blot results which demonstrates the results from studies conducted to demonstrate Reg Receptor expression and purification utilizing 293T cells that were transfected with Reg Receptor expression plasmid DNA. FIG. 7A demonstrates the use of sodium dodecyl sulfate polyacrylamide gel electrophoresis (SDS-PAGE) to illustrate the high purity of the Reg Receptor. FIGS. 7B and 7C are the Western blot results showing that the purified protein is the Reg Receptor using the antibody to the FLAG epitope (FIG. 7B) and Reg Receptor (CD104) (FIG. 7C).

FIG. 8 is a graph which shows the 7-amino acid Regenerative peptide, the 8-amino acid Regenerative peptide and the 9-amino acid Regenerative peptide all bind directly to Reg Receptor.

FIG. 9 is an image of a Western Blot which demonstrates a confirmation study to evaluate the translocation of the Reg Receptor from the cytoplasmic membrane of human extra-islet exocrine tissue. The Western blot analysis identified he presence of the Reg receptor on the cytoplasmic membrane and the movement of the Reg Receptor from the cytoplasmic membrane through the cytoplasm to the nucleus in the presence of the Regenerative peptides 7aa (SEQ ID NO: 3), Baa (SEQ ID NO: 4) and 9aa (SEQ ID NO: 5).

FIG. 10 is a table which demonstrates the results showing upregulated transcription factors stimulated by the Regenerative peptides acting through their receptor in an in-vivo mouse study of injected with peptides SEQ ID NO: 4, SEQ ID NO:5, SEQ ID NO: 8, SEQ ID NO:1 and SEQ ID NO:16

FIG. 11 is a set of sequences which demonstrate the 7-amino acid human Reg sequences (SEQ ID NO: 3) that has 100% homology with Reg sequences found in other mammals including chimpanzee, rat, mouse, golden hamster, guinea pig, rabbit, pig, sheep, cow, white-cheeked gibbon, Sumatran orangutan, Lowland gorilla, white-tufted-eared marmoset, European domestic ferret, which have not been described in the prior art as being a peptide which has not been described in the prior art as being a peptide that interacts with the Reg receptor for the generation of new neurons, cardiac myocytes, nephrons and hepatocytes

FIG. 12 is a set of sequence which demonstrate the 8-amino acid human Reg sequence (SEQ ID NO: 4) that has 100% homology with Reg sequences found in other mammals including chimpanzee, rat, golden hamster, guinea pig, rabbit, pig, sheep, cow, white-cheeked gibbon, Sumatran orangutan, Lowland gorilla, white-tufted-eared marmoset, which have not been described in the prior art as being a peptide that interacts with the Reg receptor that can generate for generation of as a peptide for generation of new neurons, cardiac myocytes, nephrons and hepatocytes.

FIG. 13 is a set of sequence which demonstrate the 9-amino acid human Reg sequences (SEQ ID NO: 14) that has 100% homology with Reg sequences found in other mammals including chimpanzee, rat, mouse, golden hamster, white-cheeked gibbon, Lowland gorilla, and white-tufted-eared marmoset, which have not been described in the prior art as being a peptide that interacts with the Reg receptor that can generate for generation of being a peptide for generation of new neurons, cardiac myocytes, nephrons and hepatocytes.

FIG. 14 is a set of sequence which demonstrates the 20-amino acid human sequence has been identified within the Reg receptor that has 100% homology with Reg receptor sequences found in other mammals including chimp, rat, mouse, guinea pig, rabbit, dog, cow, opossum, galago, white-cheeked gibbon, Sumatran orangutan, macaque, Lowland gorilla, white-tufted eared marmoset, horse and Tasmanian devil from which stimulatory antibodies and small molecules can be generated to interact with this receptor site resulting in new neurons, hepatocytes, myocytes and nephrons.

FIGS. 15A and B are microscopic images which show immunofluorescent staining of Reg receptor on the cell surface of human pancreatic exocrine ductal cells. In utilizing Cy3 immunofluorescent staining of Reg receptor in human pancreatic ductal cells in standard medium, there is immunofluorescent staining of Reg receptor, which is well-defined at the cell borders indicating surface expression of Reg receptor on the cytoplasmic membrane of cells (FIG. 15A). FIG. 15B demonstrates the difference in Reg receptor staining when cells are exposed to Reg.

FIG. 16 shows the protocol for the stability testing in human plasma for the Regenerative Peptides SEQ ID NO: 1, SEQ ID NO: 8, SEQ ID NO:3 and SEQ ID NO:16

FIG. 17 is a table which shows the Regenerative Peptides SEQ ID NO: 1, SEQ ID NO: 8, SEQ ID NO:3, SEQ ID NO:16 and the incubation time points used in the stability testing in human plasma.

FIG. 18A is a table which shows the results of the Regenerative Peptides SEQ ID NO:1 incubated in human plasma.

FIG. 18B is a table which shows the results of the Regenerative Peptides SEQ ID NO: 8 incubated in human plasma.

FIG. 18C is a graph which shows the results of the Regenerative Peptides SEQ ID NO:1 and SEQ ID NO: 8 incubated in human plasma. SEQ ID NO: 8 is significantly more resistant to proteolysis in human plasma compared to SEQ ID NO:1

FIG. 19A is a table which shows the results of the Regenerative Peptides SEQ ID NO:16 incubated in human plasma.

FIG. 19B is a table which shows the results of the Regenerative Peptides SEQ ID NO:3 incubated in human plasma.

FIG. 19C is a graph which shows the results of the Regenerative Peptides SEQ ID NO:16 and SEQ ID NO: 3 incubated in human plasma. SEQ ID NO: 16 is significantly more resistant to proteolysis in human plasma compared to SEQ ID NO:3

### DETAILED DESCRIPTION OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Over the past decade, the regenerating gene (Reg or REG) family has emerged among many species, including humans, as a potential key initiating factor in the process of new cell formation within an organ following acute injury, including the brain, heart and liver. Although Reg is typically expressed during embryogenesis when organs are being populated for the first time and downregulated after fetal development, the Reg genes are upregulated within the brain, pancreas, heart and liver when there is acute injury with the formation of new neurons, islets, cardiac myocyte, renal nephron and hepatocyte neogenesis within the injured organs (Levetan. J Diabetes. 2010 June; 2(2):76-84, Levetan Endocr Pract. 2008; 14(9). After fetal development, the Reg genes are usually undetectable, but are upregulated in response to acute organ injury.

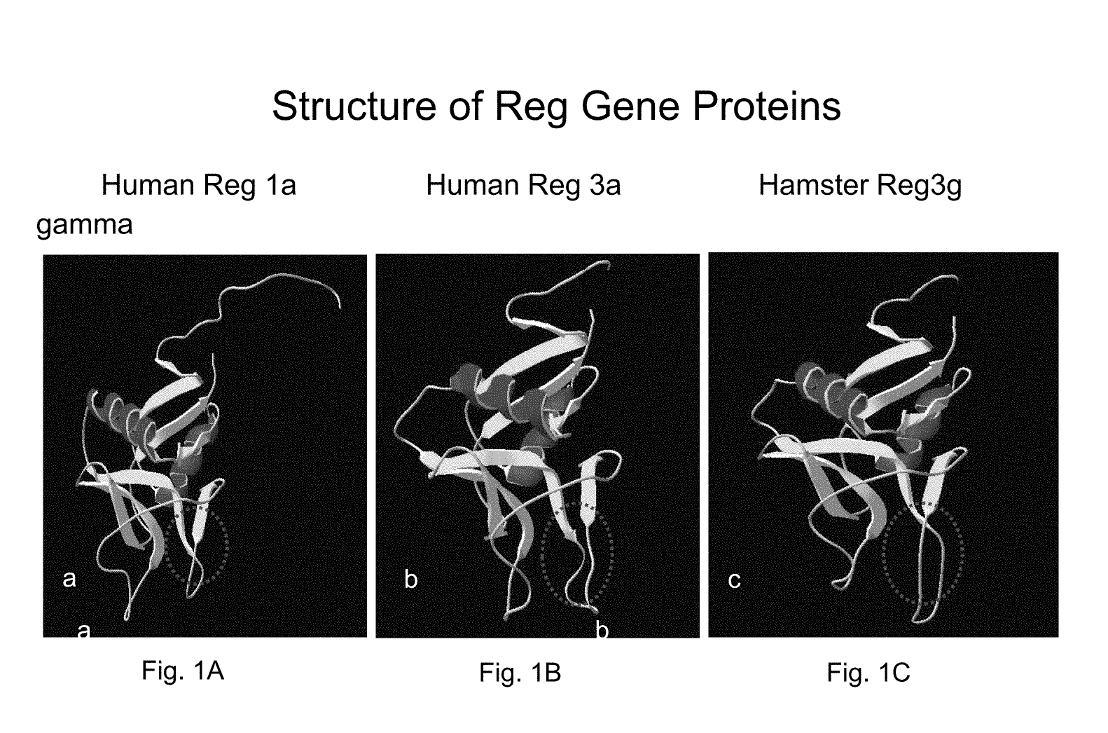

FIGS. 1A-C are illustrations showing the similarities and differences between three dimensional structures of human Reg1a, Reg3a and the Hamster Reg3 gamma peptides based on their primary -amino acid sequences and by SwissProt folding algorithms. FIG. 1A shows the three dimensional structure for Human Reg1a. FIG. 1B shows the three dimensional structure for Reg3a. FIG. 1C shows the three dimensional structure for the hamster Reg3gamma. The circled region of the protein that contains the Regenerative peptides described in this invention, which bind to Reg Receptor. Red circled arm indicates the bioactive region and homologous sequence of the Reg proteins that is the binding arm to the Reg receptor.

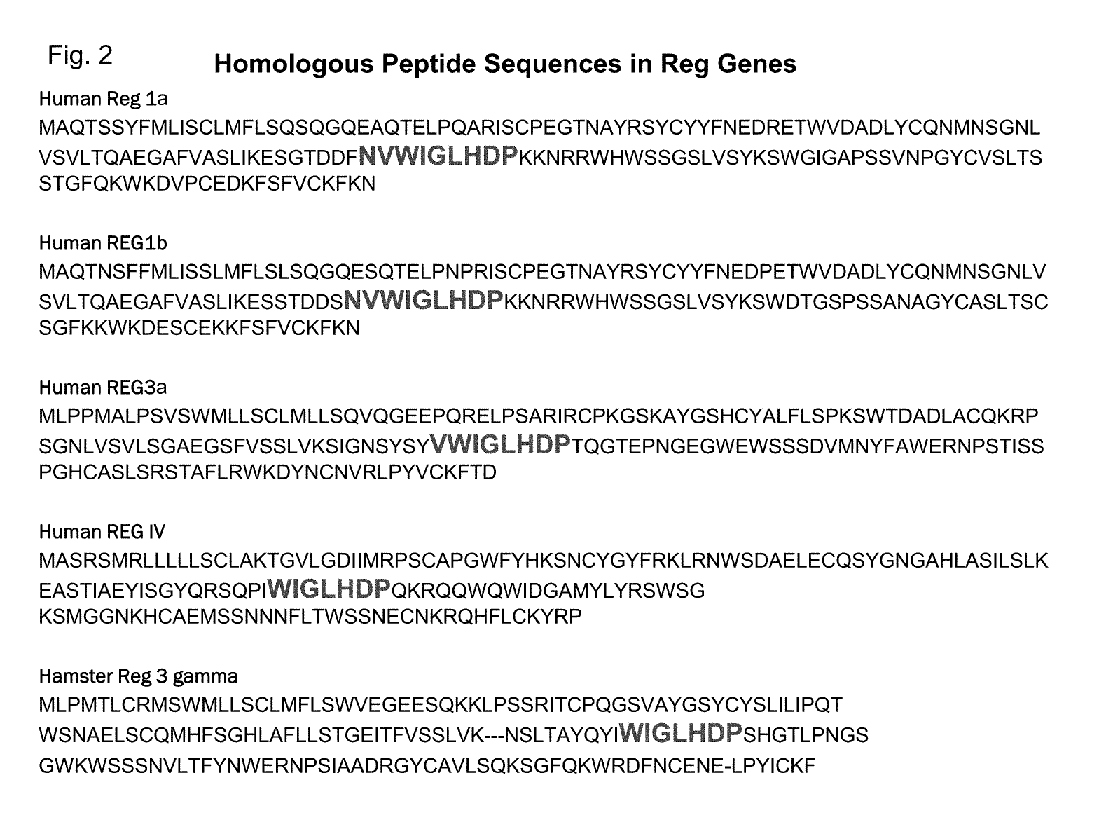

FIG. 2 is an alignment of the 166-amino acid sequence for Reg1a, the 166-amino acid sequence for Reg1b, the 175-amino acid sequence for Reg3 alpha, the 158-amino acid sequence for Reg4, and the mammalian Reg3gamma protein have been shown in the prior art to interact with the Reg receptor, resulting in downstream generation of new adult cells. FIG. 2 shows the identification of the 100% homologous sequence within the human Reg 1a, Reg1b, Reg3a, Reg4 and the Hamster Reg3gamma peptides that is located within the binding arm of the protein that binds to the Reg Receptor. This exact sequence is also found in the human, chimpanzee, rat, mouse, golden hamster, guinea pig, rabbit, pig, sheep, cow, white-cheeked gibbon, Sumatran orangutan, Lowland gorilla, white-tufted-eared marmoset, and European domestic ferret.

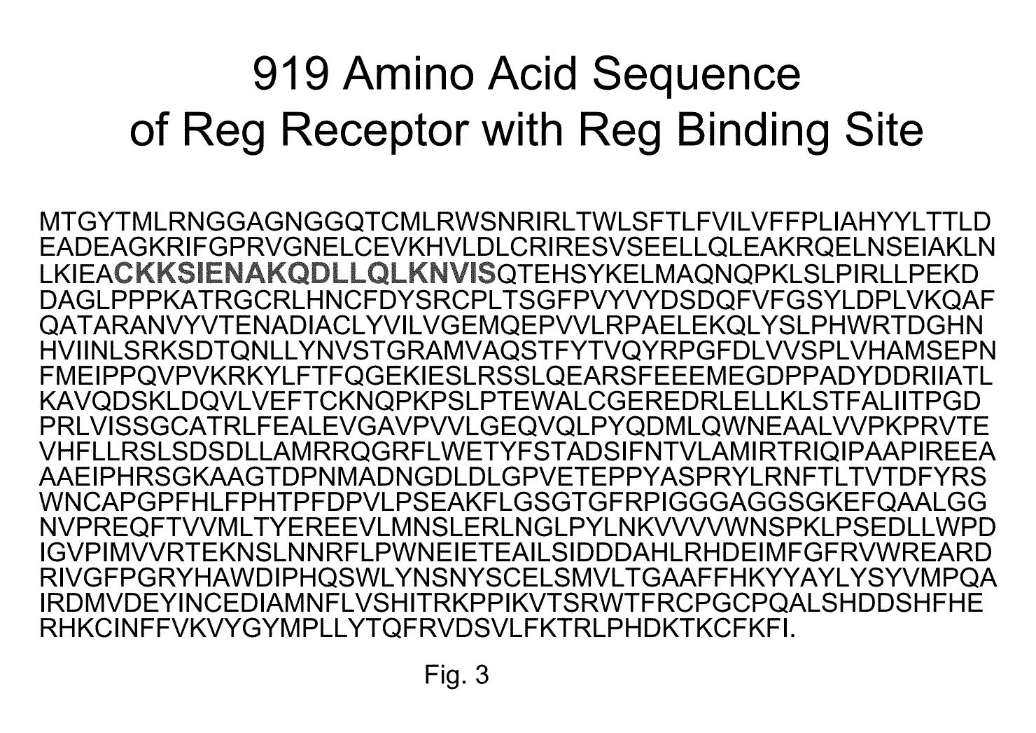

FIG. 3 shows the 919-amino acid Reg Receptor (SEQ ID NO: 7) and the 20-amino acid domain (SEQ ID NO: 6) in red that was identified in this discovery that is a binding domain for the Regenerative Peptides, and from which stimulatory antibodies to this region of the receptor have been generated.

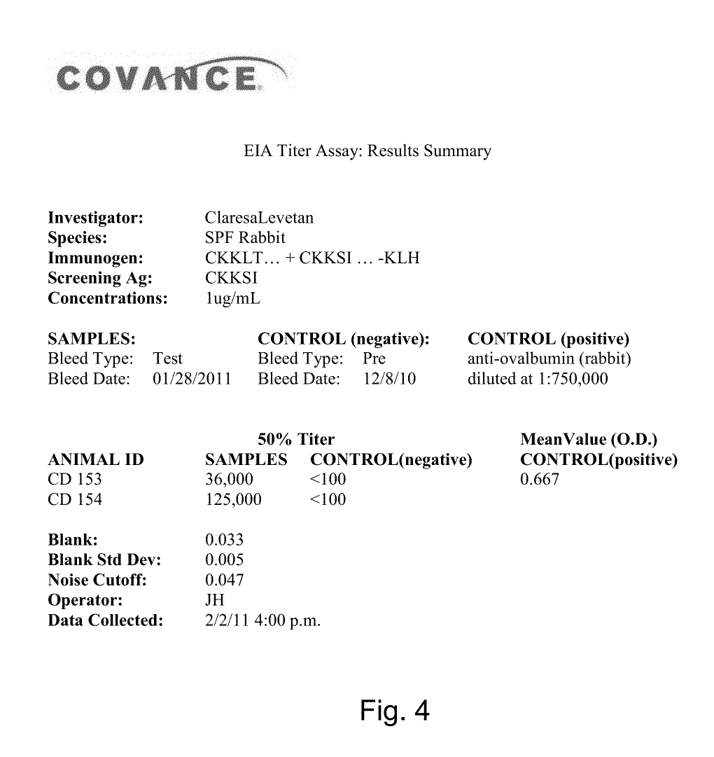

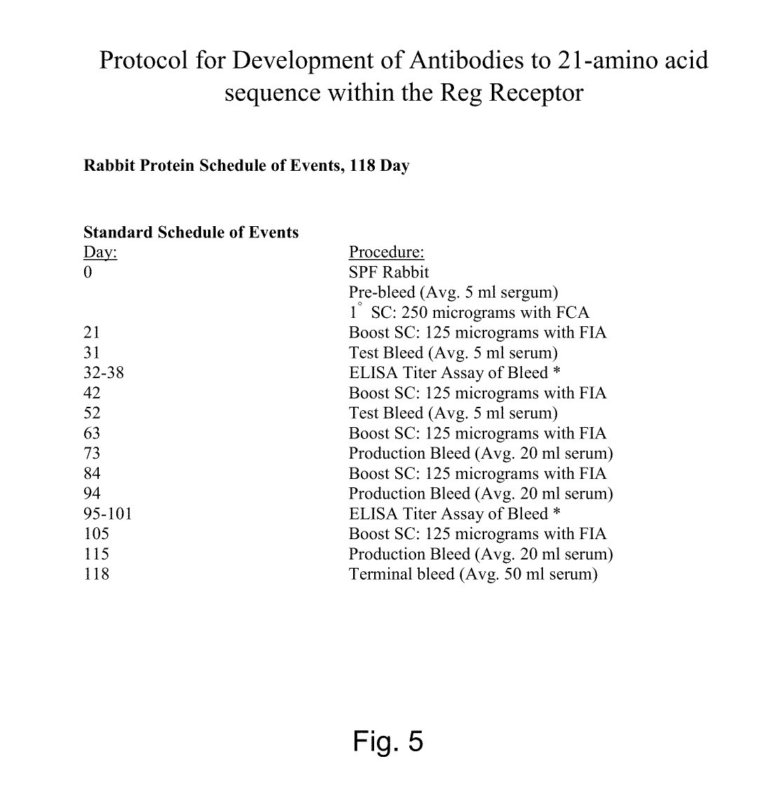

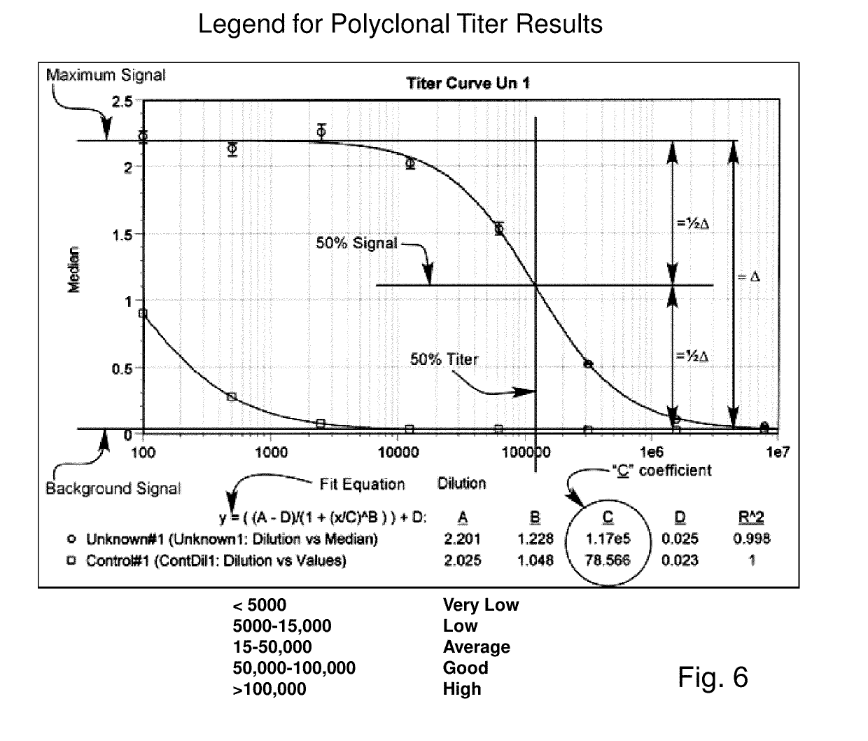

FIGS. 4-6 were from studies undertaken by this inventor to develop stimulatory antibodies to the 20-amino acid bioactive region of the 919-amino acid Reg Receptor/EXTL-3 to accelerate the downstream regulation of transcription factors to develop new cells. Progression of beta cell formation by stimulating potential binding sites on the Reg Receptor. For the production of in stimulatory antibodies and identification and confirmation of the 20-amino acid binding region of EXTL-3, many sequences were evaluated within the N-terminal portion of the Reg receptor/Receptor within amino acids 1-332 of (SEQ ID NO:7) to find the region to which antibodies were produced, which this inventor identifies as the binding domain of the Reg peptides. Consistently, in Enzyme ImmunoAssay studies measuring titers from peptide sequences within SEQ ID NO:7, resulted in very high polyclonal antibodies being raised to a 20-amino acid Reg receptor sequence (SEQ ID NO: 8) (amino acids 117-136). The results of the Enzyme ImmunoAssay are summarized in FIG. 4. FIG. 5 demonstrates the standard protocol used for development of antibodies to peptide regions within Reg receptor. Data sets were taken from the bleed after the day 0 and day 21 boosts. The animals were injected with a peptide of SEQ ID NO: 6 and were conjugated to keyhole limpet hemocyanin (KLH). The screening antigen is not conjugated to KLH so that the response solely to the peptide and not to KLH can be identified. The 50% titer is a dilution value where the signal is half-way between the peak and the baseline, so the higher the dilution value (titer), the greater the response to the antigen. The positive control is an internal control that was generated from ovalbumin antibodies in rabbit. At a dilution of 1:750,000, the absorbance fell within a range of 0.45 to 0.9. In the case of the response to SEQ ID NO: 6, there was a high response (FIG. 4). The test bleed taken 31 days after the day 0 and 21 day boosts for CD 153 showed a 50% titer of 36,000 which is an average response, and CD 154 showed a 50% titer of 125,000 which is a high response according to the titer reference range shown in FIG. 6. Studies are underway to evaluate the efficacy of the antibody generated with and without the presence of Reg peptide demonstrating that the antibodies to the Reg peptide interaction with Reg receptor to generate new neurons, cardiac myocytes, nephrons and hepatocytes.

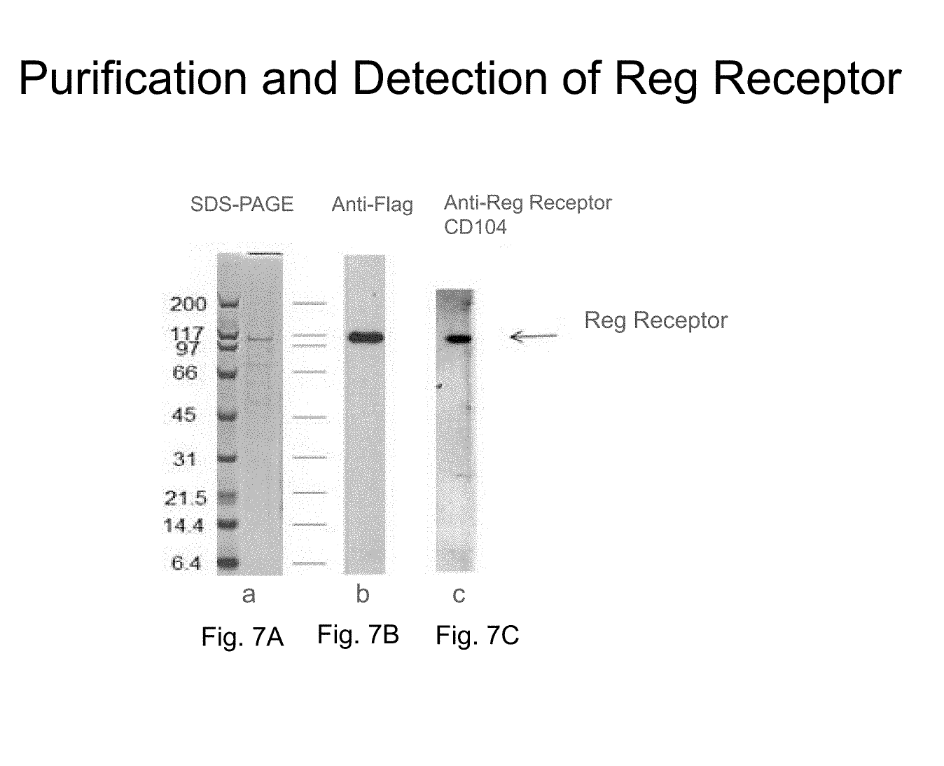

FIGS. 7A-C demonstrates the results from studies conducted to demonstrate Reg Receptor expression and purification utilizing 293T cells that were transfected with Reg Receptor expression plasmid DNA. Cells were collected after 72 hours. The Reg Receptor was tagged with FLAG epitope and FLAG resin was utilized to purify out the Reg Receptor. As shown in FIG. 7C, the Reg Receptor was highly purified. The Reg Receptor was purified by Anti-Flag M2 affinity gel. Target protein was confirmed by 4-12% SDS-PAGE and Western-blot. FIG. 7A demonstrates the use of sodium dodecyl sulfate polyacrylamide gel electrophoresis (SDS-PAGE) to illustrate the high purity of the Reg Receptor. FIGS. 7B and 7C are the Western blot results showing that the purified protein is the Reg Receptor using the antibody to the Reg Receptor (CD104). The Reg Receptor is shown in FIG. 7C to be highly purified. To then demonstrate the direct binding of Regenerative Peptides to the Reg Receptor, the purified Reg receptor was coated onto 96 well plate by using bicarbonate coating buffer, pH 9.6; 4° C. overnight at concentration 3 ug/ml, 100 ul per well. Plates were coated overnight coated plate and washed three times with 0.5× TBST and blocked with 3% BSA and rotated at room temperature for 1 hour. After blocking, plates were washed three times with 0.5× TBST. Peptides were then diluted with TBST buffer and added into wells in duplicate then left to bind at room temperature for 1 hour. After washing three times, 100 ng/ml strep-HRP was added into plate at 100 ul/well, and rotated at room temperature for one hour. ABST reagents were warmed to room temperature, mixed immediately before using. Then 100 ul was added to each well and read after 25 minutes reaction and absorbance was evaluated at 405 nm by a Spectramax M5 plate reader. The purified Reg Receptor was coated on plates. Then plates were blocked with BSA solution. Subsequently, the Regenerative peptides were added into the wells, and HRP-straptavidin and its substrates were added into the wells to reveal the interaction between Receptor and the peptide.

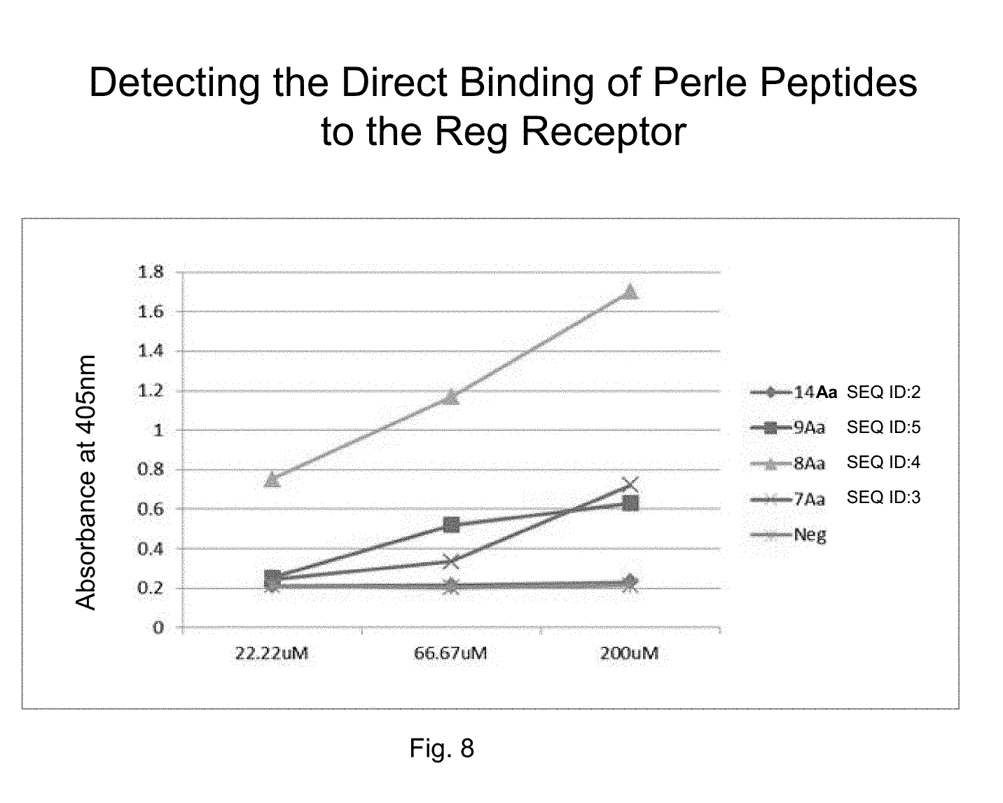

FIG. 8 shows the 7-amino acid Reg peptide (SEQ ID NO: 3), the 8-amino acid Reg peptide (SEQ ID NO: 4) and the 9-amino acid Reg peptide all show binding (SEQ ID NO: 5) to the Reg Receptor. The scrambled control peptide did not bind to Reg Receptor (FIG. 8).

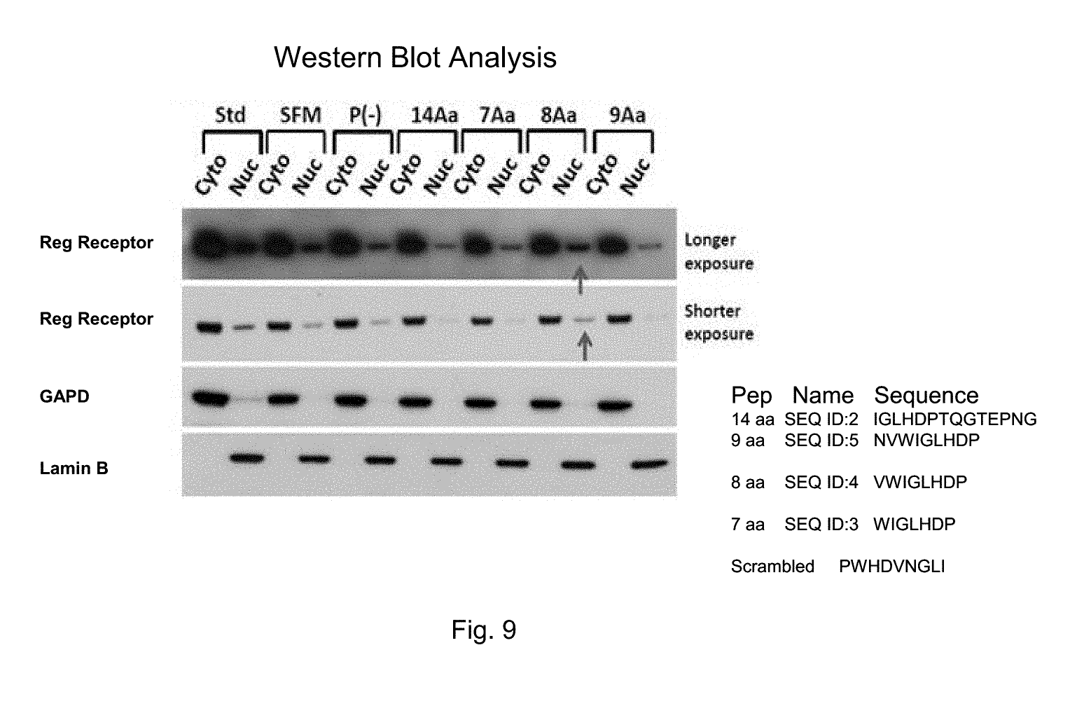

FIG. 9 demonstrates a confirmation study utilizing Western blot analysis to evaluate the translocation of the Reg Receptor from the cytoplasmic membrane of human extra-islet exocrine tissue inclusive of ductal cells containing progenitor. Western blot analysis identified the presence of the Reg receptor on the cytoplasmic membrane and the movement of the Reg Receptor from the cytoplasmic membrane through the cytoplasm to the nucleus in the presence of the Regenerative peptides (30 minute treatment). Cytoplasmic extracts were obtained in 10 mM HEPES (pH 8.0), 1 mM EDTA, 1.5 mM MgCl2, 10 mM KCl, 0.5 mM DTT, 200 mM sucrose and 0.5% Nonidet P-40. Nuclear extracts were obtained in 20 mM HEPES (pH 7.9), 0.75 mM MgCl2, 210 mM NaCl, 50 mM KCl, 1 mM EDTA, 10% glycerol, and 0.5 mM DTT. Both extraction buffers contained 0.5 mM PMSF, 1 μg/ml leupeptin, 1 μg/ml aprotinin, 2.5 mM Na4P2O7, 1 mM β-glycerophosphate, and 1 mM Na3VO4. Reg Receptor extracts were size fractionated on SDS-polyacrylamide gels and transferred to nitrocellulose. After blocking in 3% milk in Tris-buffered saline (pH 7.4), blots were sequentially incubated with rabbit anti-human Reg Receptor antibody overnight at 4° C. and appropriate horseradish peroxidase-conjugated secondary antibody. Secondary signals were developed with chemiluminescence substrate and analyzed by autoradiography.

Fractions and quality control utilized the fractions with two antibodies, GAPDH as a cytosol molecule and Lamin B is a nuclear molecule. Both GADPH and Lamin B were demonstrated in this invention to serve as excellent controls for the nuclear and cytosolic fractions (FIG. 9). Evaluation was conducted to determine the impact and interaction of the Reg Receptor with the Regenerative peptides and the human 14-amino acid (SEQ ID NO: 2) peptide. This invention demonstrates in FIG. 9 that the 8-amino acid (SEQ ID NO: 4) Regenerative Peptide resulted in the translocation of the Reg Receptor from the cytoplasm to the nucleus both in the shorter and longer exposure times.

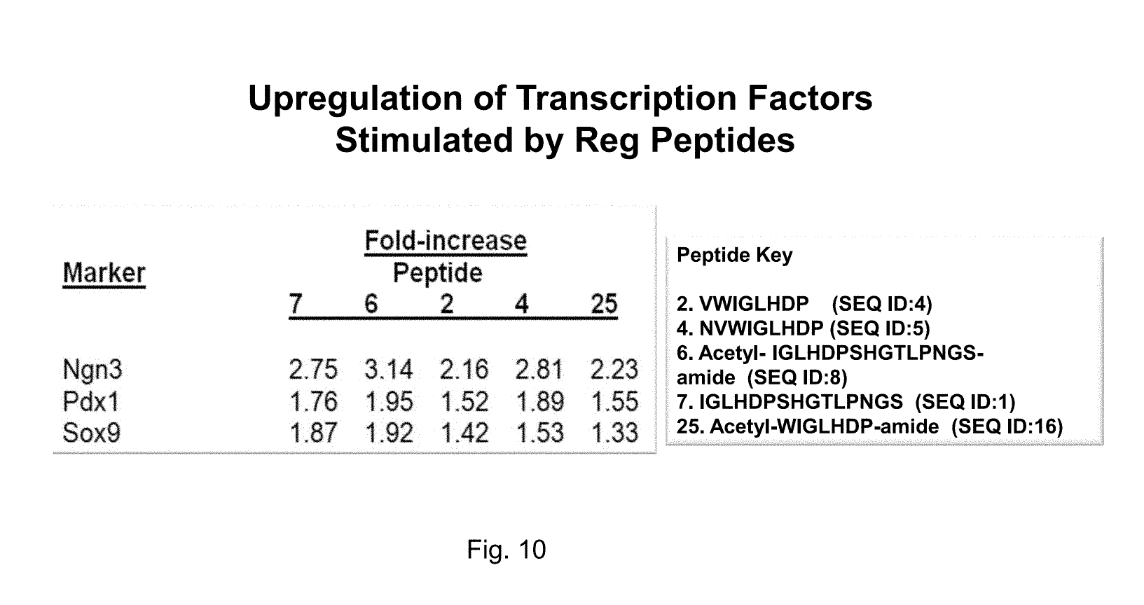

FIG. 10 demonstrates the in vivo efficacy of Reg peptides on pancreatic transcription factors NGN3, Pdx1, and Sox9 compared to uninjected and vehicle alone controls with cohorts of 10 mice (adult, age ˜8 weeks, mixed male and female) will be injected intraperitoneally with selected candidate Reg peptides at 2-3 dosages/animal daily across 2-3 time courses (an average of 60 mice/peptide tested). An additional 20 animals will serve as negative (uninjected and vehicle alone) controls. Pancreata were subjected to rapid dissection and denaturant RNA preparation followed by real-time, quantitative RT-PCR analysis of gene expression markers for beta cell development and differentiation, including Ngn3, Pdx1 and Sox9. All Reg peptides had a significant fold increase in transcription factors ranging from a 1.33 fold rise to a 3.44-fold rise compared to vehicle and control transcription factors, indicating the ability to raise pancreatic transcription factors in mice.

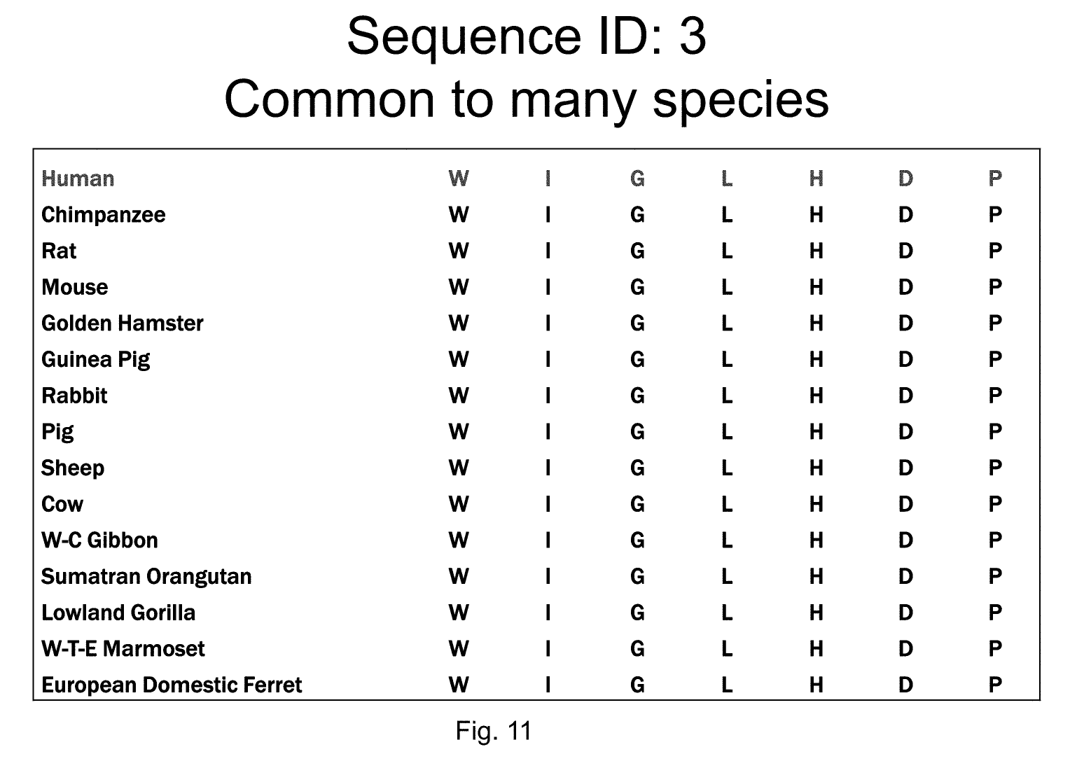

FIG. 11 shows the identification of the Regenerative Peptide 7-amino acid human Reg sequence (SEQ ID NO: 3) that has 100% homology with sequences found in other mammals including chimpanzee, rat, mouse, golden hamster, guinea pig, rabbit, pig, sheep, cow, white-cheeked gibbon, Sumatran orangutan, Lowland gorilla, white-tufted-eared marmoset, European domestic ferret which has not been described in the prior art as being a peptide that interacts with the Reg receptor that can generate new neurons, cardiac myocytes, nephrons and hepatocytes in times of injury when the Reg receptor is upregulated in such organs. This invention evaluated GenBank, Basic Local Alignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR).

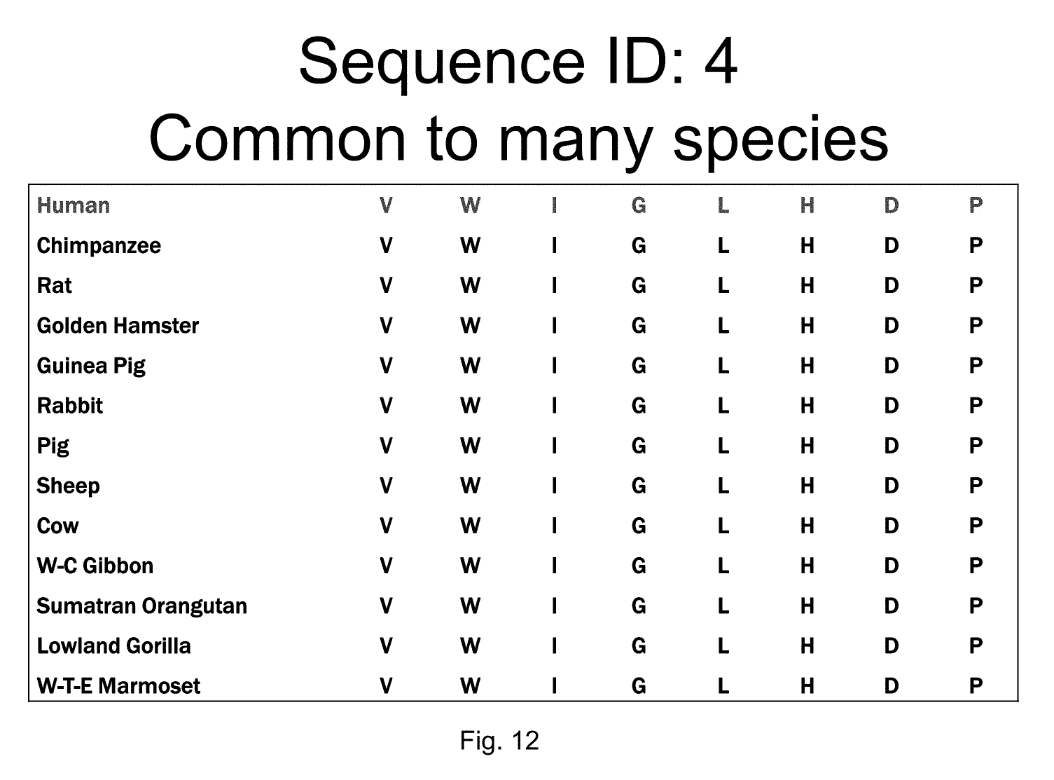

FIG. 12 shows the identification of the Regenerative Peptide 8-amino acid human Reg sequence (SEQ ID NO: 4) that has 100% homology with sequences found in other mammals including chimpanzee, rat, mouse, golden hamster, guinea pig, rabbit, pig, sheep, cow, white-cheeked gibbon, Sumatran orangutan, Lowland gorilla, and white-tufted-eared marmoset which have not been described in the prior art as being a peptide that interacts with the Reg receptor that can generate new neurons, cardiac myocytes, nephrons and hepatocytes. This invention evaluated GenBank, Basic LocalAlignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR)

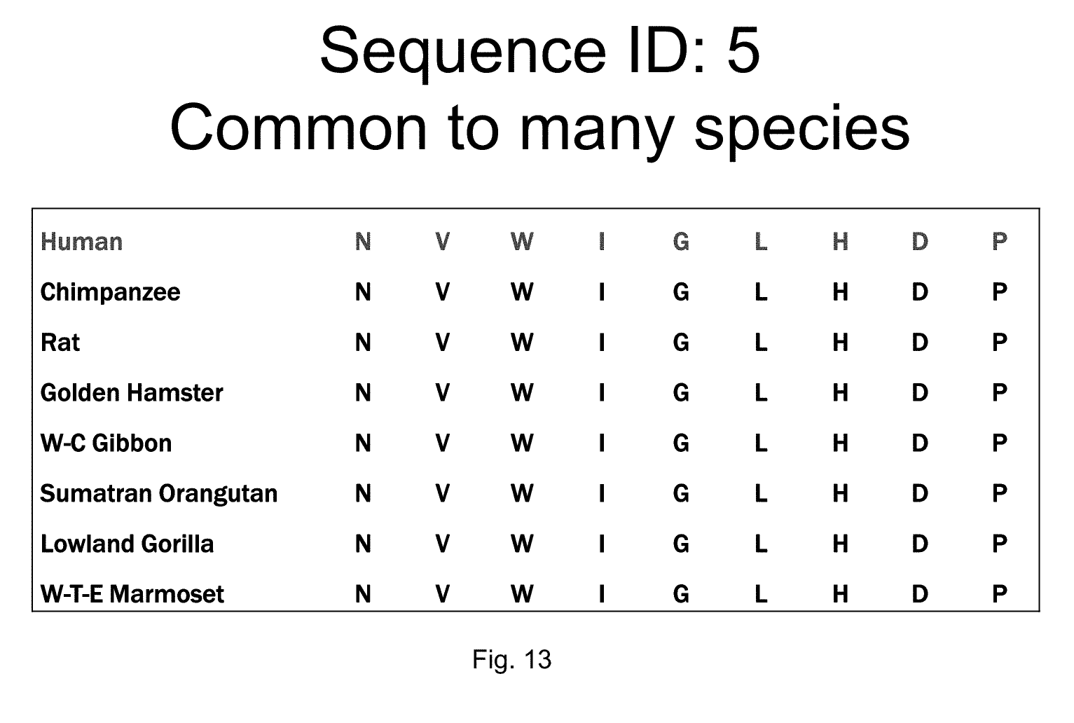

FIG. 13 shows the identification of the Regenerative Peptide 9-amino acid human Reg sequence (SEQ ID NO: 5) that has 100% homology with sequences found in other mammals including chimpanzee, rat, golden hamster, white-cheeked gibbon, Sumatran orangutan, Lowland gorilla, white-tufted-eared marmoset, which have not been described in the prior art as being a peptide that interacts with the Reg receptor that can generate for generation of new neurons, cardiac myocytes, nephrons and hepatocytes due to its ability to bind to This invention evaluated GenBank, Basic Local Alignment Search Tool (BLAST) algorithm and UniProtKB, which produced by the UniProt Consortium which consists of groups from the European Bioinformatics Institute (EBI), the Swiss Institute of Bioinformatics (SIB) and the Protein Information Resource (PIR).

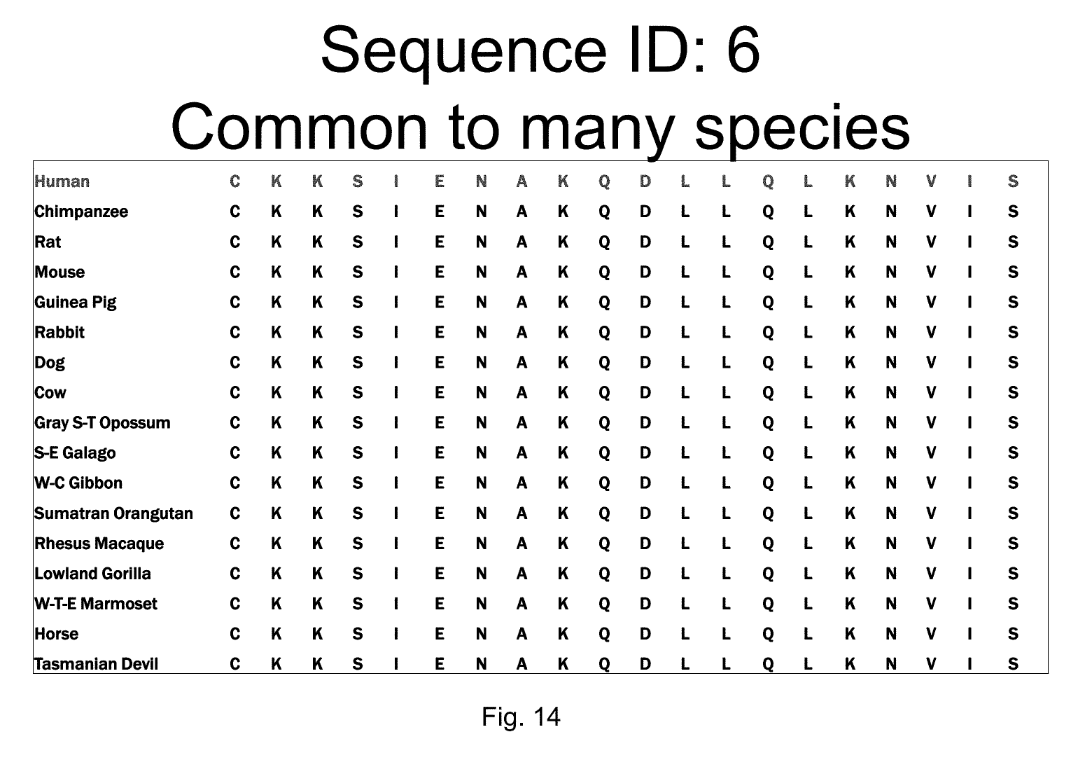

FIG. 14 demonstrates the 20-amino acid human sequence (SEQ ID NO: 6) that has been identified within the human Reg receptor that has 100% homology with Reg receptor sequences found in other mammals including chimp, rat, mouse, guinea pig, rabbit, dog, cow, opossum, galago, white-cheeked-gibbon, Sumatran orangutan, macaque, Lowland gorilla, white-tufted eared marmoset, horse and Tasmanian devil from which stimulatory antibodies and small molecules to this receptor site can be generated to result in new neurons, hepatocytes, myocytes and nephrons. This invention evaluated GenBank, Basic Local Alignment Search Tool (BLAST) algorithm and UniProtKB which produced by the UniProt Consortium which consists of groups from the European Bioinformatics Institute (EBI), the Swiss Institute of Bioinformatics (SIB) and the Protein Information Resource (PIR).

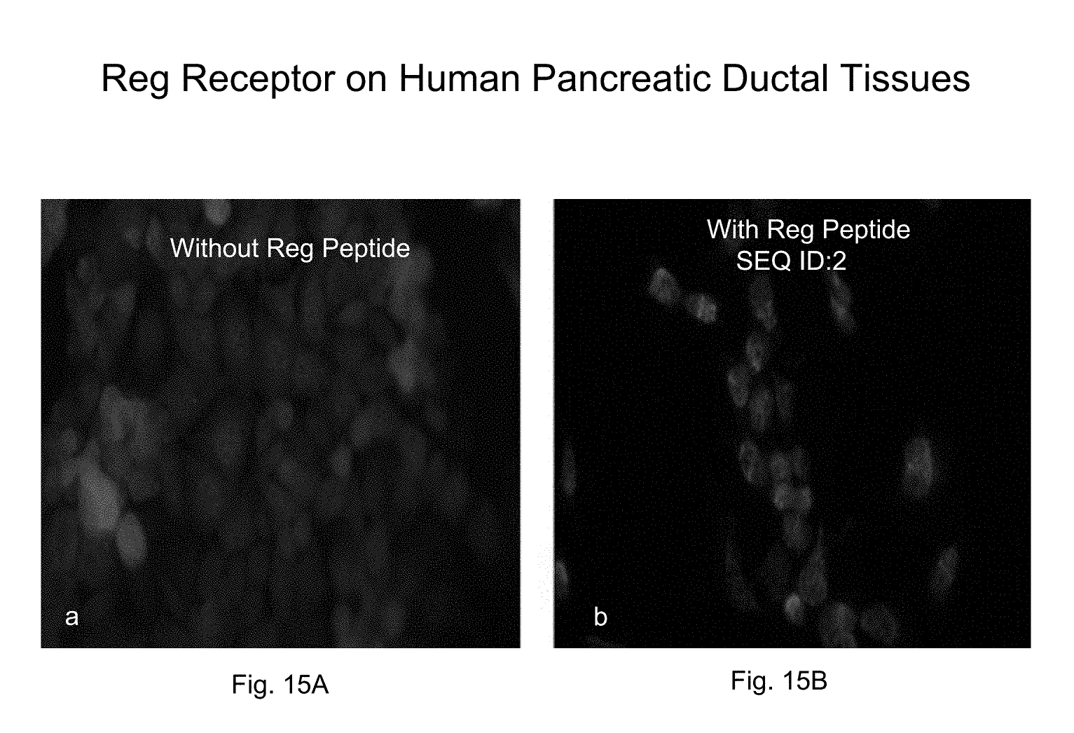

FIGS. 15A and 15B shows immunofluorescent staining of Reg receptor on the cell surface of human pancreatic exocrine ductal cells. In utilizing Cy3 immunofluorescent staining of Reg receptor in human pancreatic ductal cells in standard medium, there is immunofluorescent staining of Reg receptor, which is well-defined at the cell borders indicating surface expression of Reg receptor on the cytoplasmic membrane of cells (FIG. 15A). FIG. 15B demonstrates the difference in Reg receptor staining when cells are exposed to Reg peptide (SEQ ID NO: 2).

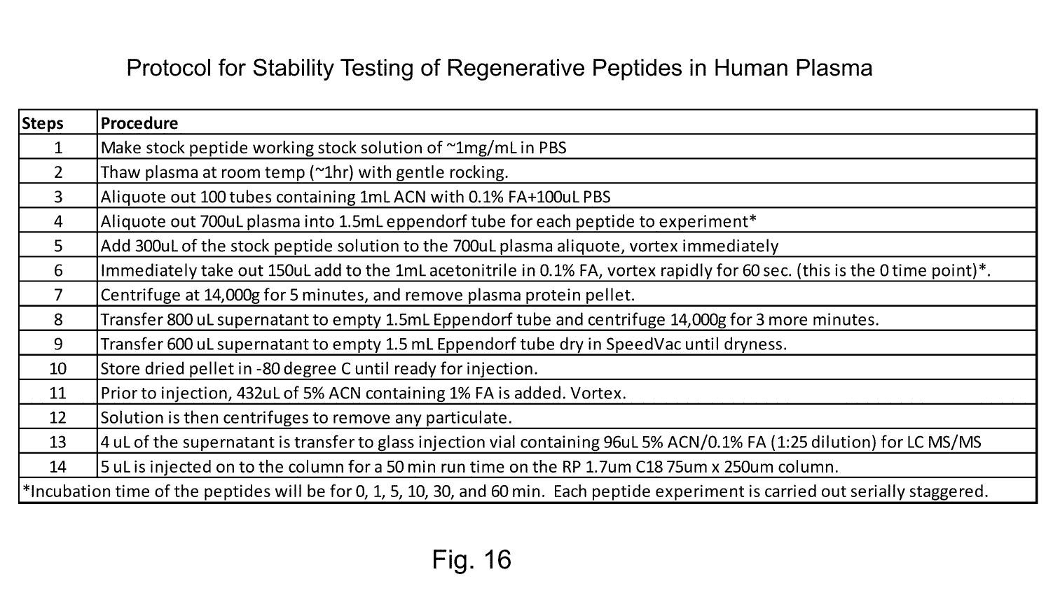

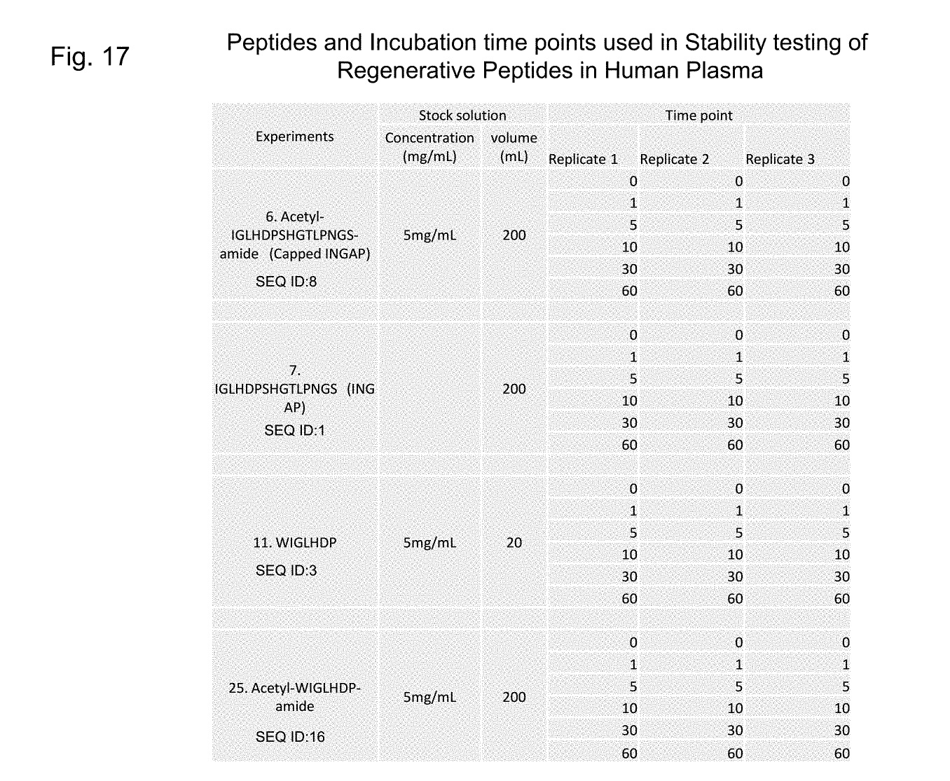

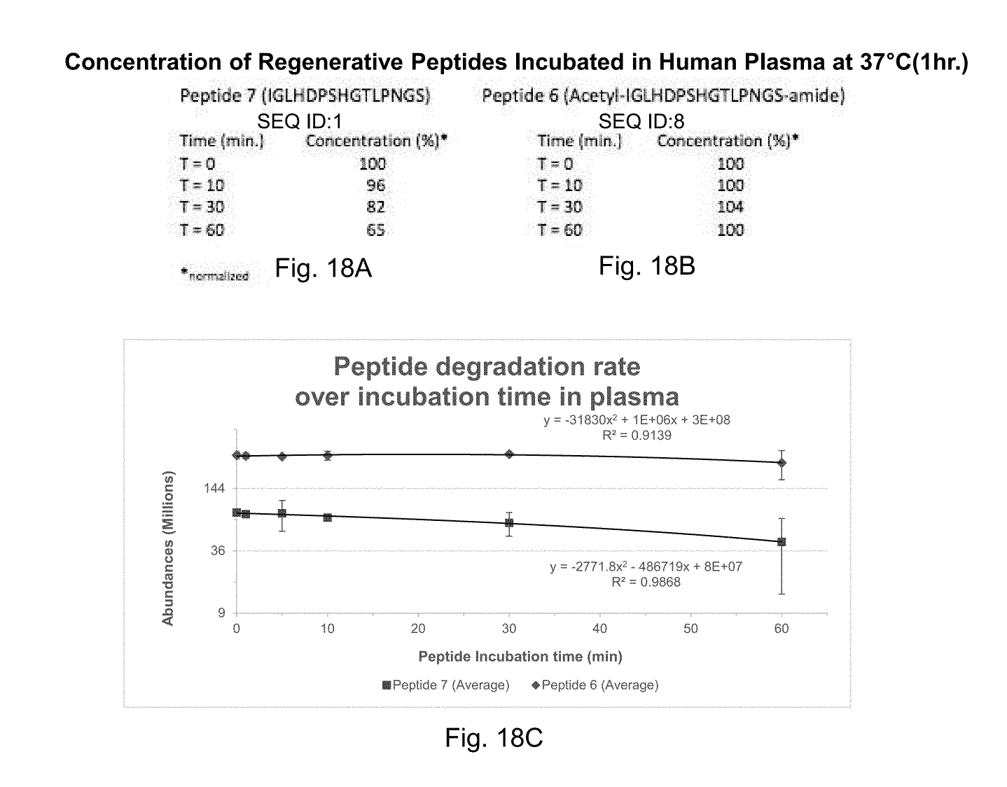

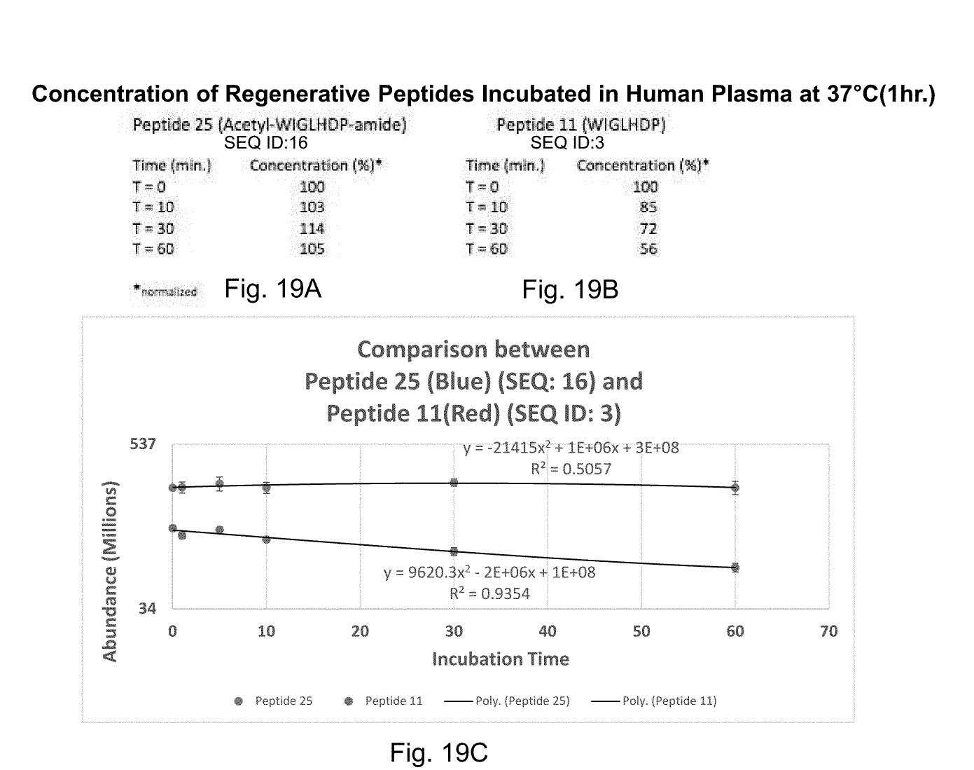

FIGS. 16-19C demonstrate the methods for stability testing of Regenerative Peptides SEQ ID NO: 1, SEQ ID NO: 8, SEQ ID NO: 3 and SEQ ID NO:16 and the incubation time points used in the stability testing in human plasma. FIG. 16 shows the protocol for the stability testing in human plasma for the Regenerative Peptides SEQ ID NO: 1, SEQ ID NO: 8, SEQ ID NO:3 and SEQ ID NO:16. FIG. 17. shows Peptides and Incubation time points used in Stability testing of Regenerative Peptides in Human Plasma. FIGS. 18A-C shows the results of the Regenerative Peptides SEQ ID NO:1 and SEQ ID NO: 8 incubated in human plasma. SEQ ID NO: 8 is significantly more resistant to proteolysis in human plasma compared to SEQ ID NO:1. FIGS. 19A-C show the results of the Regenerative Peptides SEQ ID NO: 16 and SEQ ID NO: 3 incubated in human plasma. SEQ ID NO: 16 is significantly more resistant to proteolysis in human plasma compared to SEQ ID NO: 3.

### BRIEF DESCRIPTION OF THE SEQUENCES

As used herein, “Peg-40KD-maleimide” includes:

Methoxy-PEG-(CH2)3NHCO(CH2)2-MAL, Mw 40,000

Chemical Name: α-[3-(3-Maleimido-1-oxopropyl)amino]propyl-ω-methoxy, polyoxyethylene

CAS#: 883993-35-9

As used herein, 3-Mpa=3-mercaptopropionic acid, CAS#: 107-96-0

SEQ ID NO: 1

This is the 15-amino acid peptide found within the mammalian Reg protein

Ile-Gly-Leu-His-Asp-Pro-Ser-His-Gly-Thr-Leu-Pro-
Asn-Gly-Ser

SEQ ID NO: 2

This is the 14-amino acid peptide found within the human Reg protein

Ile-Gly-Leu-His-Asp-Pro-Thr-Gln-Gly-Thr-Glu-Pro-
Asn-Gly

SEQ ID NO: 3

This is the 7-amino acid peptide found within the human and mammalian Reg protein

Trp-Ile-Gly-Leu-His-Asp-Pro

SEQ ID NO: 4

This is the 8-amino acid peptide found within the human and mammalian Reg protein

Val-Trp-Ile-Gly-Leu-His-Asp-Pro

SEQ ID NO: 5

This is the 9-amino acid peptide found within the human and mammalian Reg protein

Asn-Val-Trp-Ile-Gly-Leu-His-Asp-Pro

SEQ ID NO: 6

This is a 20-amino acid peptide found within the human Reg receptor also known as EXTL-3

Cys-Lys-Lys-Ser-Ile-Glu-Asn-Ala-Lys-Gln-Asp-Leu-
Leu-Gln-Leu-Lys-Asn-Val-Ile-Ser

SEQ ID NO: 7

Is the 919-amino acid human Reg receptor, also known as EXTL-3, exostosin-like 3 (public accession number NP_001431).

MTGYTMLRNGGAGNGGQTCMLRWSNRIRLTWLSFTLFVILVFFPLIAHYY
LTTLDEADEAGKRIFGPRVGNELCEVKHVLDLCRIRESVSEELLQLEAKR
QELNSEIAKLNLKIEACKKSIENAKQDLLQLKNVISQTEHSYKELMAQNQ
PKLSLPIRLLPEKDDAGLPPPKATRGCRLHNCFDYSRCPLTSGFPVYVYD
SDQFVFGSYLDPLVKQAFQATARANVYVTENADIACLYVILVGEMQEPVV
LRPAELEKQLYSLPHWRTDGHNHVIINLSRKSDTQNLLYNVSTGRAMVAQ
STFYTVQYRPGFDLVVSPLVHAMSEPNFMEIPPQVPVKRKYLFTFQGEKI
ESLRSSLQEARSFEEEMEGDPPADYDDRIIATLKAVQDSKLDQVLVEFTC
KNQPKPSLPTEWALCGEREDRLELLKLSTFALIITPGDPRLVISSGCATR
LFEALEVGAVPVVLGEQVQLPYQDMLQWNEAALVVPKPRVTEVHFLLRSL
SDSDLLAMRRQGRFLWETYFSTADSIFNTVLAMIRTRIQIPAAPIREEAA
AEIPHRSGKAAGTDPNMADNGDLDLGPVETEPPYASPRYLRNFTLTVTDF
YRSWNCAPGPFHLFPHTPFDPVLPSEAKFLGSGTGFRPIGGGAGGSGKEF
QAALGGNVPREQFTVVMLTYEREEVLMNSLERLNGLPYLNKVVVVWNSPK
LPSEDLLWPDIGVPIMVVRTEKNSLNNRFLPWNEIETEAILSIDDDAHLR
HDEIMFGFRVWREARDRIVGFPGRYHAWDIPHQSWLYNSNYSCELSMVLT
GAAFFHKYYAYLYSYVMPQAIRDMVDEYINCEDIAMNFLVSHITRKPPIK
VTSRWTFRCPGCPQALSHDDSHFHERHKCINFFVKVYGYMPLLYTQFRVD
SVLEKTRLPHDKTKCFKFI.

SEQ ID NO: 8

This is the optimization of the mammalian 15-amino acid Reg peptide by blocking with an n-terminal acetyl group and a c-terminal amide group.

Ac--Ile-Gly-Leu-His-Asp-Pro-Ser-His-Gly-Thr-Leu-
Pro-Asn-Gly-Ser-NH2

SEQ ID NO: 9

This is a pegylated version of the mammalian 15-amino acid Reg peptide

Ac--Ile-Gly-Leu-His-Asp-Pro-Ser-His-Gly-Thr-Leu-
Pro-Asn-Gly-Ser (Peg-40KD-maleimide)-NH2

SEQ ID NO:10

This is a pegylated version of the mammalian 15-amino acid Reg peptide

Peg-40KD-maleimide-3-Mpa-Ile-Gly-Leu-His-Asp-Pro-
Ser-His-Gly-Thr-Leu-Pro-Asn-Gly-Ser-NH2 (3-Mpa = 3-
mercaptopropionic acid)

SEQ ID NO:11

This is a cyclized versions of the mammalian 15-amino acid Reg peptide to include but are not limited to:

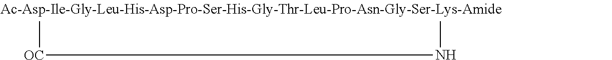

Cyclic amide bond between side chain of Asp on position 1 and Lys on position 17

SEQ ID NO: 12

This is the optimization of the 14-amino acid human Reg peptide by blocking with an n-terminal acetyl group and a c-terminal amide group.

Ac--Ile-Gly-Leu-His-Asp-Pro-Thr-Gln-Gly-Thr-Glu-
Pro-Asn-Gly--NH2

SEQ ID NO: 13

This is a pegylated version of the human 14-amino acid Reg peptide

Ac--Ile-Gly-Leu-His-Asp-Pro-Thr-Gln-Gly-Thr-Glu-
Pro-Asn-Gly (Peg-40KD-maleimide)-NH2

SEQ ID NO:14

This is a pegylated version of the human 14-amino acid Reg peptide

Peg-40KD-maleimide-3-Mpa-Ile-Gly-Leu-His-Asp-Pro-
Thr-Gln-Gly-Thr-Glu-Pro-Asn-Gly-NH2 (3-Mpa-3-
mercaptopropionic acid)

SEQ ID NO:15

Cyclized versions of the human 14-amino acid Reg peptide to include but are not limited to:

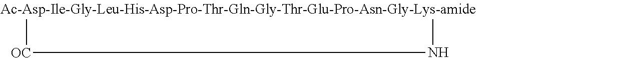

Cyclic amide bond between side chain of Asp on position 1 and Lys on position 16

SEQ ID NO: 16

This is the optimization of the 7-amino acid human and mammalian Reg peptide by blocking with an n-terminal acetyl group and a c-terminal amide group.

Ac--Trp-Ile-Gly-Leu-His-Asp-Pro--NH2

SEQ ID NO: 17

This is a pegylated version of the human and mammalian 7-amino acid Reg peptide

Ac-Trp-Ile-Gly-Leu-His-Asp-Pro (Peg-40KD-
maleimide)-NH2

SEQ ID NO:18

This is a pegylated version of the human and mammalian 7-amino acid Reg peptide

Peg-40KD-maleimide-3-Mpa-Trp-Ile-Gly-Leu-His-Asp-
Pro-NH2 (3-Mpa = 3-mercaptopropionic acid)

SEQ ID NO:19

Cyclized versions of the human and mammalian 7-amino acid Reg 3 peptide to include but are not limited to:

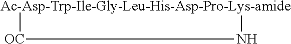

Cyclic amide bond between side chain of Asp on position 1 and Lys on position 9

SEQ ID NO: 20

This is the optimization of the 8-amino acid human and mammalian Reg peptide by blocking with an n-terminal acetyl group and a c-terminal amide group.

Ac-Val-Trp-Ile-Gly-Leu-His-Asp-Pro--NH2

SEQ ID NO: 21

This is a pegylated version of the human and mammalian 8-amino acid Reg protein

Ac-Val-Trp-Ile-Gly-Leu-His-Asp-Pro (Peg-40KD-
maleimide)-NH2

SEQ ID NO:22

This is a pegylated version of the human and mammalian 8-amino acid Reg peptide

Peg-40KD-maleimide-3-Mpa-Val-Trp-Ile-Gly-Leu-His-
Asp-Pro-NH2 (3-Mpa = 3-mercaptopropionic acid)

SEQ ID NO:23

Cyclized versions of the human and mammalian 8-amino acid Reg peptide to include but are not limited to:

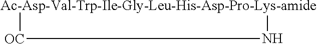

Cyclic amide bond between side chain of Asp on position 1 and Lys on position 10

SEQ ID NO: 24

This is the optimization of the human and mammalian 9-amino acid Reg peptide by blocking with an n-terminal acetyl group and a c-terminal amide group.

Ac-Asn-Val-Trp-Ile-Gly-Leu-His-Asp-Pro--NH2

SEQ ID NO: 25

This is a pegylated version of the human and mammalian 9-amino acid Reg peptide

Ac-Asn-Val-Trp-Ile-Gly-Leu-His-Asp-Pro (Peg-40KD-
maleimide)-NH2

SEQ ID NO:26

This is a pegylated version of the human and mammalian 9-amino acid Reg peptide

Peg-40KD-maleimide-3-Mpa-Asn-Val-Trp-Ile-Gly-Leu-
His-Asp-Pro-NH2 (3-Mpa = 3-mercaptopropionic acid)

SEQ ID NO:27

Cyclized versions of the human and mammalian 9-amino acid Reg peptide to include but are not limited to:

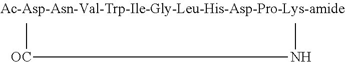

Cyclic amide bond between side chain of Asp on position 1 and Lys on position 11

The Reg peptides may be produced through recombinant molecular biology techniques or solid phase synthesis techniques. Recombinant molecular biology techniques include those described in Molecular Cloning: A Laboratory Manual, Green and Sanbrook, 2012. Solid-phase synthesis techniques are described in Merrifield, in J. Am. Chem. Soc., 15:2149-2154 (1963), M. Bodanszky et al., (1976) Peptide Synthesis, John Wiley & Sons, 2d Ed.; Kent and Clark-Lewis in Synthetic Peptides in Biology and Medicine, p. 295-358, eds. Alitalo, K., et al. Science Publishers, (Amsterdam, 1985); as well as other reference works known to those skilled in the art such. A summary of peptide synthesis techniques may be found in J. Stuart and J. D. Young, Solid Phase Peptide Synthelia, Pierce Chemical Company, Rockford, Ill. (1984), which is incorporated herein by reference. The synthesis of peptides by solution methods may also be used, as described in The Proteins, Vol. II, 3d Ed., p. 105-237, Neurath, H. et al., Eds., Academic Press, New York, N.Y. (1976). Appropriate protective groups for use in such syntheses will be found in the above texts, as well as in J. F. W. McOmie, Protective Groups in Organic Chemistry, Plenum Press, New York, N.Y. (1973), which is incorporated herein by reference. In general, these synthetic methods involve the sequential addition of one or more amino acid residues or protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine. Block synthesis techniques may also be applied to both the solid phase and solution methods of peptide synthesis. Rather than sequential addition of single amino acid residues, preformed blocks comprising two or more amino acid residues in sequence are used as either starting subunits or subsequently added units rather than single amino acid residues. Alternative or additional peptide synthesis methods and techniques can be found in Peptide Chemistry: A Practical Textbook: 2nd Edition, Miklos Bodanszky, 1993.

Reg peptides of the invention may also be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting peptides is accomplished using conventional procedures, such as preparative HPLC using gel permeation, partition and/or ion exchange chromatography. The choice of appropriate matrices and buffers are well known in the art and so are not described in detail herein.

Inert polymer molecules such as high molecular weight polyethyleneglycol (PEG) can be attached to a peptide of this disclosure or an analog or derivative thereof with or without a multifunctional linker either through site-specific conjugation of the PEG to the N- or C-terminus of the protein or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules. Unreacted PEG can be separated from peptide-PEG conjugates by size-exclusion or by ion-exchange chromatography.

Protocols for blocking peptides with acetyl and amide groups are known in the art and can be found in a number of protein protocol textbooks known in the art. Specific examples include those published in Methods in Molecular Biology, Vol. 35: Peptide Synthesis Protocols, Chapter 8: Site-Specific Chemical Modification Procedures, Edited by M W Pennington and B M Dunn, 1994, as well as U.S. Pat. Nos. 4,708,934, 5,503,989, U.S Patent Application Publication No. US 20060127995. Alternative or additional protein modification procedures can be found in Peptide Chemistry: A Practical Textbook: 2nd Edition, Miklos Bodanszky, 1993. Further, peptide cyclization (Davies, J. Peptide Sci. 9: 471-501 (2003)) and pegylation (Roberts, Advanced Drug Delivery Reviews (2002) 54:459-476 and Veronese, Biomaterials (2001) 22(5):405-17) methods have been reviewed. Protocols for creating maleimide-activated PEG constructs may be found in Schumacher et al., In Situ Maleimide Bridging of Disulfides and a New Approach to Protein PEGylation, Bioconjugate Chem., 2011, 22 (2), pp 132-136, Doherty et al., Site-Specific PEGylation of Engineered Cysteine Analogs of Recombinant Human Granulocyte-Macrophage Colony-Stimulating Factor, Bioconjug Chem. 2005; 16(5): 1291-1298, US Patent Application Publication No. 20090298746 A1, European Patent No. EP 1881850 B1, European Patent No. EP 2178900 B1.

### Embodiments

Embodiments of the invention include but are not limited to the following:

Embodiment 1A: Regenerative Peptides which comprise the following 7-15-amino acid Reg sequences and 7-15-amino acid optimized Reg Peptides sequences from the human and mammalian Reg peptides that are utilized for both direct production of nerve cells, cardiac myocytes, liver cells and kidney cells via in vivo formation by the usage of the 7-15 optimized and native peptides in times of acute loss of cells from the brain, spinal cord, heat, kidney and liver with such peptides used for the formation of new neurons, including motor neurons, cardiac myocytes, liver hepatocytes and kidney nephrons and include: SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 and may be delivered to a patient in need of new neurons, myocytes, hepatocytes, and nephrons by Routes of delivery of therapies described include, but are not limited to oral, intravenous, intra-arterial, subcutaneous delivery and intrathecal delivery and through the spinal canal for generation central neurons and for spinal cord injuries. Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions and delivered to the renal artery via the femoral artery with a small puncture in the groin. A catheter is inserted through the artery and directed toward the renal artery. Once the catheter is positioned in the artery supplying blood to the kidney, above sequences cam be injected into the renal artery for the formation of new nephrons.

Embodiment 2A: Regenerative Peptides which comprise the following 7-15-amino acid Reg sequences and 7-15-amino acid optimized Reg Peptides sequences from the human and mammalian Reg peptides that are utilized for production of nerve cells, cardiac myocytes, liver cells and kidney cells via ex vivo formation by the usage of the 7-15 optimized and native peptides generated outside of the body using ex vivo cells. Which include, but are not limited neural stems cells, tissue stem cells, mesenchymal stem cells, totipotent embyronic stem cells, multipotent stem cells, pluripotent stem cells, including embryonic cells, adult somatic stem cells, human adult bone-marrow derived stem cells, umbilical cord stems cells, human amniotic membrane-derived mesenchymal cells, mammalian stem cells, cord cells, ectodermal stem or any other tissue or cell types that can be transformed by human and mammalian 7-15-amino acid Reg peptide sequences into functional neurons, myocytes, hepatocytes and nephrons. from which Reg peptides can transform such tissue into adult functioning cells for usage in times of acute loss of cells from the brain, spinal cord, heart, kidney and liver with such peptides used for the formation of new neurons, including motor neurons, cardiac myocytes, liver hepatocytes and kidney nephrons and include: SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 and then neurons, myocytes, nephrons, hepatocytes may be delivered to a patient in need of new neurons, myocytes, hepatocytes, and nephrons by Routes of delivery of therapies described include, but are not limited to oral, intravenous, intra-arterial, subcutaneous delivery and intrathecal delivery and through the spinal canal for generation central neurons and for spinal cord injuries. Also, therapy may be given by organ specific targeting and may include direct administration liver via the umbilical and hepatic artery, femoral or radial arterial delivery to the heart or directly to the arteries supplying the heart, as is done in cardiac catheterizations, and intrathecal delivery and through the spinal canal for central nervous system as is done for antibiotic delivery for central nervous system infections and for chemotherapy delivery for central lesions and delivered to the renal artery via the femoral artery with a small puncture in the groin. A catheter is inserted through the artery and directed toward the renal artery. Once the catheter is positioned in the artery supplying blood to the kidney, above sequences cam be injected into the renal artery for the formation of new nephrons. This modality of delivery includes, but is not limited to: oral, intravenous, subcutaneously, intra-arterial, intrathecal and into the spinal column and targeted delivery to the brain, spinal column, kidney liver or delivered directly or indirectly to the brain, spinal column, heart and liver and may include targeted therapy to the given organ, which may be delivered orally, intravenously, intra-arterially, intrathecally or directly into the spinal column at the site of injury.

Embodiment 3A: Peptidomimetics, small molecules and stimulatory antibodies used in vivo to stimulate the 20-amino acid region on the 919-amino acid Reg Receptor/EXTL-3 (SEQ: ID 6) to generate of new neurons, cardiac myocytes, nephrons and hepatocytes. Administration of peptidomimetics includes formulations that stimulate SEQ ID 6 on the EXTL-3 receptor resulting in formation of new cardiac myocytes, nephrons, hepatocytes and neurons. This modality of delivery includes, but is not limited to: oral, intravenous, subcutaneously, intra-arterial, intrathecal and into the spinal column and targeted delivery to the brain, spinal column, kidney liver or delivered directly or indirectly to the brain, spinal column, heart and liver and may include targeted therapy to the given organ, which may be delivered orally, intravenously, intra-arterially, intrathecally or directly into the spinal column at the site of injury.

Embodiment 4A: Peptidomimetics, small molecules and stimulatory antibodies used ex vivo to stimulate the 20-amino acid region on the 919-amino acid Reg Receptor/EXTL-3 (SEQ: ID 6) to generate of new neurons, cardiac myocytes, nephrons and hepatocytes to transform neural stems cells, tissue stem cells, mesenchymal stem cells, totipotent embyronic stem cells, multipotent stem cells, pluripotent stem cells, including embryonic cells, adult somatic stem cells, human adult bone-marrow derived stem cells, umbilical cord stems cells, human amniotic membrane-derived mesenchymal cells, mammalian stem cells, cord cells, ectodermal stem or any other tissue or cell types into functional neurons, myocytes, hepatocytes and nephrons. The modality of delivery of peptidomimetics, small molecules and antibodies to stimulate (SEQ ID 6) includes, but is not limited to: oral, intravenous, subcutaneously, intra-arterial, intrathecal and into the spinal column and targeted delivery to the brain, spinal column, kidney liver or delivered directly or indirectly to the brain, spinal column, heart and liver and may include targeted therapy to the given organ, which may be delivered orally, intravenously, intra-arterially, intrathecally or directly into the spinal column at the site of injury.

Embodiment 5A: SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27 are used to generate neurons, hepatocytes, myocytes and nephrons in vivo and ex vivo and may be used in conjunction with other medications used to treat heart disease, liver disease, neurological diseases and renal diseases.

Embodiment 6A: Peptidomimetics, small molecules and stimulatory antibodies used ex vivo to stimulate the 20-amino acid region on the 919-amino acid Reg Receptor/EXTL-3 (SEQ: ID 6) to generate of new neurons, cardiac myocytes, nephrons and hepatocytes either in vivo or ex-vivo and may be used in conjunction with other medications used to treat heart disease, liver disease, neurological diseases and renal diseases.

Embodiment 1B: An isolated or modified peptide having an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

Embodiment 2B: A pharmaceutical formulation comprising the peptide of embodiment 1B.

Embodiment 3B: The pharmaceutical formulation of embodiment 2B, wherein the formulation is a soluble liposome or nanoparticle preparation.

Embodiment 4B: The pharmaceutical formulation of embodiment 2B, wherein the formulation comprises a targeting agent for targeted administration to heart, brain, spinal column, liver, or kidney.

Embodiment 5B: A method of treating a subject in need of one more differentiated cells or tissue types, comprising administering to the subject a peptide having Reg Receptor binding activity, wherein the amount of peptide is effective for forming differentiated cells or tissues in the subject in vivo.

Embodiment 6B: The method of embodiment 5B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

Embodiment 7B: The method of embodiment 5B, wherein the one or more cell or tissue types are heart, liver, brain, spinal column, or kidney cells or tissues.

Embodiment 8B: The method of embodiment 5B, wherein the peptide is administered directly to the heart, liver, brain, spinal cord or kidney of a subject.

Embodiment 9B: The method of embodiment 5B, wherein the peptide is administered by way of intravenous, subcutaneous, intra-arterial, or intrathecal delivery.

Embodiment 10B: The method of embodiment 5B, wherein the subject has a condition selected from the group consisting of heart disease, myocardial infarction, stroke, acute brain injury, neurodegenerative disease, spinal cord injury, peripheral neuropathy, acute and chronic kidney disease and liver disease.

Embodiment 11B: The method of transforming progenitor cells to differentiated tissue cells, comprising:

culturing a plurality of progenitor cells ex vivo; and

contacting the progenitor cells with a peptide having Reg Receptor binding activity,

wherein the amount of peptide is effective for transforming progenitor cells to differentiated cells or tissues in culture.

Embodiment 12B: The method of embodiment 11B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

Embodiment 13B: The method of embodiment 11B, wherein the progenitor cells are selected from the group consisting of neural stems cells, tissue stem cells, mesenchymal stem cells, totipotent embyronic stem cells, multipotent stem cells, pluripotent stem cells, including embryonic cells, adult somatic stem cells, human adult bone-marrow derived stem cells, umbilical cord stems cells, human amniotic membrane-derived mesenchymal cells, mammalian stem cells, cord cells, and ectodermal stem cells.

Embodiment 14B: The method of embodiment 11B, wherein the differentiated tissue cells are selected from the group consisting of brain, spinal cord, heart, kidney and liver tissue cells.

Embodiment 15B: A method of treating a subject in need of one more differentiated cells or tissues, the method comprising:

culturing a plurality of progenitor cells ex vivo;

contacting the progenitor cells with a peptide having Reg Receptor binding activity, wherein the amount of peptide is effective for transforming the progenitor cells to differentiated cells or tissues in culture; and

administering the one or more differentiated cells or tissues to the subject.

Embodiment 16B. The method of embodiment 15B, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

Embodiment 17B. The method of embodiment 15B, wherein the progenitor cells are selected from the group consisting of neural stems cells, tissue stem cells, mesenchymal stem cells, totipotent embyronic stem cells, multipotent stem cells, pluripotent stem cells, including embryonic cells, adult somatic stem cells, human adult bone-marrow derived stem cells, umbilical cord stems cells, human amniotic membrane-derived mesenchymal cells, mammalian stem cells, cord cells, and ectodermal stem cells.

Embodiment 18B. The method of embodiment 15B, wherein the differentiated cells or tissues are selected from the group consisting of brain, spinal cord, heart, kidney and liver cells or tissues.

Embodiment 19B. The method of embodiment 15B, wherein the differentiated cells or tissues are administered directly to the heart, liver, brain, spinal cord or kidney of a subject.

Embodiment 20B. The method of embodiment 15B, wherein the subject has a condition selected from the group consisting of heart disease, myocardial infarction, stroke, acute brain injury, neurodegenerative disease, spinal cord injury, peripheral neuropathy, acute and chronic kidney disease and liver disease.

### EXAMPLES

The following examples serve to further illustrate the invention and should not be used to limit the invention.

### Example 1

A patient presents with an acute myocardial infarction as evidenced by EKG and cardiac isoenzymes. The patient undergoes cardiac catheterization and prior to injection of contrast is given 60 mg of SEQ ID NO: 8, which will be delivered into the myocardium and bind to the upregulated EXTL-3/Reg receptor in the location of the acute injury.

### Example 2

A patient suffers a neck injury from being thrown from a horse with the spinal cord severed at Cervical Disk 7 and has immediate paralysis. Sixty mg of SEQ ID NO: 8, is delivered directly via injection to the severed cord under fluoroscopy and delivered every day for 14 days.

### Example 3

A patient undergoes resection of the liver for removal of a large metastatic lesion from colon cancer. An oral hepatic targeted nanoparticle encapsulation of SEQ ID NO: 8 in a dosage of 60 mg and is given to the patient twice daily.

### Example 4

A patient with marked renal insufficiency with a creatinine of 4.0 is given 60 mg of SEQ ID NO: 8 delivered renal artery via the femoral artery with a small puncture in the groin. A catheter is inserted through the artery and directed toward the renal artery. Once the catheter is positioned in the artery supplying blood to the kidney, SEQ ID NO: 8 is injected into the renal artery. This is repeated monthly.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure including published patents, published patent applications, books, and journal articles are each individually incorporated by reference herein in their entireties and as such are intended to provide an efficient way of supplementing the enabling disclosure of this invention as well as provide background detailing the level of ordinary skill in the art.

1. An isolated or modified peptide having an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

2. A pharmaceutical formulation comprising the peptide of claim 1.

3. The pharmaceutical formulation of claim 2, wherein the formulation is a soluble liposome or nanoparticle preparation.

4. The pharmaceutical formulation of claim 2, wherein the formulation comprises a targeting agent for targeted administration to heart, brain, spinal column, liver, or kidney.

5. A method of treating a subject in need of one or more differentiated cells or tissue types, comprising administering to the subject a peptide having Reg Receptor binding activity, wherein the amount of peptide is effective for forming differentiated cells or tissues from progenitor cells in the subject in vivo.

6. The method of claim 5, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

7. The method of claim 5, wherein the one or more differentiated cells or tissue types are heart, liver, brain, spinal column, or kidney cells or tissues.

8. The method of claim 5, wherein the peptide is administered directly to the heart, liver, brain, spinal cord or kidney of a subject.

9. The method of claim 5, wherein the peptide is administered by way of intravenous, subcutaneous, intra-arterial, or intrathecal delivery.

10. The method of claim 5, wherein the subject has a condition selected from the group consisting of heart disease, myocardial infarction, stroke, acute brain injury, neurodegenerative disease, spinal cord injury, peripheral neuropathy, acute and chronic kidney disease and liver disease.

11. A method of treating a subject in need of one more differentiated cells or tissues, the method comprising:
culturing a plurality of progenitor cells ex vivo;
contacting the progenitor cells with a peptide having Reg Receptor binding activity, wherein the amount of peptide is effective for transforming the progenitor cells to differentiated cells or tissues in culture; and
administering the one or more differentiated cells or tissues to the subject.

12. The method of claim 11, wherein the peptide having Reg Receptor binding activity has an amino acid sequence selected from the group consisting of SEQ ID NO: 1, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

13. The method of claim 11, wherein the progenitor cells are selected from the group consisting of neural stems cells, tissue stem cells, mesenchymal stem cells, totipotent embyronic stem cells, multipotent stem cells, pluripotent stem cells, including embryonic cells, adult somatic stem cells, human adult bone-marrow derived stem cells, umbilical cord stems cells, human amniotic membrane-derived mesenchymal cells, mammalian stem cells, cord cells, and ectodermal stem cells.

14. The method of claim 11, wherein the differentiated cells or tissues are selected from the group consisting of brain, spinal cord, heart, kidney and liver cells or tissues.

15. The method of claim 11, wherein the differentiated cells or tissues are administered directly to the heart, liver, brain, spinal cord or kidney of a subject.

16. The method of claim 11, wherein the subject has a condition selected from the group consisting of heart disease, myocardial infarction, stroke, acute brain injury, neurodegenerative disease, spinal cord injury, peripheral neuropathy, acute and chronic kidney disease and liver disease.


SEQUENCE LISTING (27 sequences)

SEQ ID NO: 1 - protein, 15 aa, Mesocricetus auratus
IGLHDPSHGTLPNGS

SEQ ID NO: 2 - protein, 14 aa, Homo sapiens
IGLHDPTQGTEPNG

SEQ ID NO: 3 - protein, 7 aa, Homo sapiens
WIGLHDP

SEQ ID NO: 4 - protein, 8 aa, Homo sapiens
VWIGLHDP

SEQ ID NO: 5 - protein, 9 aa, Homo sapiens
NVWIGLHDP

SEQ ID NO: 6 - protein, 20 aa, Homo sapiens
CKKSIENAKQDLLQLKNVIS

SEQ ID NO: 7 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI

SEQ ID NO: 8 - protein, 15 aa, Artificial
  synthetic
  ACETYLATION (1)..(1)
  AMIDATION (15)..(15)
IGLHDPSHGTLPNGS

SEQ ID NO: 9 - protein, 15 aa, Artificial
  synthetic
  ACETYLATION (1)..(1)
  PEGYLATION (15)..(15): conjugation with 40-kDa poly (ethylene glycol) (PEG)-maleimide
  AMIDATION (15)..(15)
IGLHDPSHGTLPNGS

SEQ ID NO: 10 - protein, 15 aa, Artificial
  synthetic
  PEGYLATION (1)..(1): conjugation with 40-kDa poly (ethylene glycol) (PEG)-maleimide-3-mercaptopropionic acid
  AMIDATION (15)..(15)
IGLHDPSHGTLPNGS

SEQ ID NO: 11 - protein, 17 aa, Artificial
  synthetic
  MISC_FEATURE: Cyclic amide bond between side chain of Asp on position 1 and Lys on position 17
  ACETYLATION (1)..(1)
  AMIDATION (17)..(17)
DIGLHDPSHGTLPNGSK

SEQ ID NO: 12 - protein, 14 aa, Artificial
  synthetic
  ACETYLATION (1)..(1)
  AMIDATION (14)..(14)
IGLHDPTQGTEPNG

SEQ ID NO: 13 - protein, 14 aa, Artificial
  synthetic
  ACETYLATION (1)..(1)
  PEGYLATION (14)..(14): conjugation with 40-kDa poly (ethylene glycol) (PEG)-maleimide
  AMIDATION (14)..(14)
IGLHDPTQGTEPNG

SEQ ID NO: 14 - protein, 14 aa, Artificial
  synthetic
  PEGYLATION (1)..(1): conjugation with 40-kDa poly (ethylene glycol) (PEG)-maleimide-3-mercaptopropionic acid
  AMIDATION (14)..(14)
IGLHDPTQGTEPNG

SEQ ID NO: 15 - protein, 16 aa, Artificial
  synthetic
  MISC_FEATURE: Cyclic amide bond between side chain of Asp on position 1 and Lys on position 16
  ACETYLATION (1)..(1)
  AMIDATION (16)..(16)
DIGLHDPTQGTEPNGK

SEQ ID NO: 16 - protein, 7 aa, Artificial
  synthetic
  ACETLYATION (1)..(1)
  AMIDATION (7)..(7)
WIGLHDP

SEQ ID NO: 17 - protein, 7 aa, Artificial
  synthetic
  ACETYLATION (1)..(1)
  PEGYLATION (7)..(7): conjugation with 40-kDa poly (ethylene glycol) (PEG)-maleimide
  AMIDATION (7)..(7)
WIGLHDP

SEQ ID NO: 18 - protein, 7 aa, Artificial
  synthetic
  PEGYLATION (1)..(1): conjugation with 40-kDa poly (ethylene glycol) (PEG)-maleimide-3-mercaptopropionic acid
  AMIDATION (7)..(7)
WIGLHDP

SEQ ID NO: 19 - protein, 9 aa, Artificial
  synthetic
  MISC_FEATURE: Cyclic amide bond between side chain of Asp on position 1 and Lys on position 9
  ACETYLATION (1)..(1)
  AMIDATION (9)..(9)
DWIGLHDPK

SEQ ID NO: 20 - protein, 8 aa, Artificial
  synthetic
  ACETLYATION (1)..(1)
  AMIDATION (8)..(8)
VWIGLHDP

SEQ ID NO: 21 - protein, 8 aa, Artificial
  synthetic
  ACETYLATION (1)..(1)
  PEGYLATION (8)..(8): conjugation with 40-kDa poly (ethylene glycol) (PEG)-maleimide
  AMIDATION (8)..(8)
VWIGLHDP

SEQ ID NO: 22 - protein, 8 aa, Artificial
  synthetic
  PEGYLATION (1)..(1): conjugation with 40-kDa poly (ethylene glycol) (PEG)-maleimide-3-mercaptopropionic acid
  AMIDATION (8)..(8)
VWIGLHDP

SEQ ID NO: 23 - protein, 10 aa, Artificial
  synthetic
  MISC_FEATURE: Cyclic amide bond between side chain of Asp on position 1 and Lys on position 10
  ACETYLATION (1)..(1)
  AMIDATION (10)..(10)
DVWIGLHDPK

SEQ ID NO: 24 - protein, 9 aa, Artificial
  synthetic
  ACETYLATION (1)..(1)
  AMIDATION (9)..(9)
NVWIGLHDP

SEQ ID NO: 25 - protein, 9 aa, Artificial
  synthetic
  ACETYLATION (1)..(1)
  PEGYLATION (9)..(9): conjugation with 40-kDa poly (ethylene glycol) (PEG)-maleimide
  AMIDATION (9)..(9)
NVWIGLHDP

SEQ ID NO: 26 - protein, 9 aa, Artificial
  synthetic
  PEGYLATION (1)..(1): conjugation with 40-kDa poly (ethylene glycol) (PEG)-maleimide-3-mercaptopropionic acid
  AMIDATION (9)..(9)
NVWIGLHDP

SEQ ID NO: 27 - protein, 11 aa, Artificial
  synthetic
  MISC_FEATURE: Cyclic amide bond between side chain of Asp on position 1 and Lys on position 11
  ACETYLATION (1)..(1)
  AMIDATION (11)..(11)
DNVWIGLHDPK